(19) United States
(12) Patent Application Publication
(10) Pub. No.: US 2026/0038654 A1
(43) Pub. Date: Feb. 5, 2026

(54) ELECTRONIC MEDICAL RECORD/SOAP NOTE SYSTEM AND METHOD

(71) Applicant: ChiroUp, Inc., Swansea, IL (US)

(72) Inventors: Timothy Bertelsman, Swansea, IL (US); Brandon Steele, High Ridge, MO (US)

(21) Appl. No.: 19/277,058

(22) Filed: Jul. 22, 2025

Related U.S. Application Data
(60) Provisional application No. 63/679,456, filed on Aug. 5, 2024.

(51) Int. Cl. G16H 10/60; G06Q 40/08; G16H 10/20

(52) U.S. Cl. CPC G16H 10/60; G16H 10/20; G06Q 40/084

(57) ABSTRACT

A medical record system and method are disclosed which enables a SOAP note for a patient encounter to be generated and stored in an electronic medical record for the patient. The content of the SOAP note is initially auto-populated by the system in response to information received from the patient and the practitioner.

### CROSS-REFERENCE TO RELATED APPLICATIONS

This application claims priority to U.S. App. No. 63/679,456 filed Aug. 5, 2024, said application being incorporated herein by reference.

### STATEMENT REGARDING FEDERALLY SPONSORED RESEARCH OR DEVELOPMENT

Not Applicable.

### BACKGROUND

This application relates to electronic medical records (EMRs), and in particular, to an improved system for electronically generating Subjective, Objective, Assessment and Plan (SOAP) notes.

SOAP notes are typically used by healthcare professionals for documenting patient encounters. In accordance with common practice, a typical SOAP note includes a subjective portion, an objective portion, an assessment portion, and a plan portion, in that order sequential order. The subjective portion includes information from the patient regarding the health issues experienced by the patient (i.e., what the patient says is wrong). The second, objective, portion includes the observations of the healthcare provider made during the patient encounter. These observations can include vital signs and measurements, such as patient blood pressure, palpation observations, patient responses to the healthcare provider's questions, etc. The third, assessment, portion of the SOAP note contains the healthcare provider's diagnosis based on the subjective and objective components of the SOAP note. Finally, the plan portion includes the steps prescribed by the healthcare provider to be taken to treat the patient based on the assessment.

There are numerous systems for generating electronic medical records, including SOAP notes. The current systems generate the SOAP note in the traditional order—Subjective, observation, assessment, plan. However, this traditional order does not comport with the manner in which a healthcare provider conducts a patient encounter. In particular, the healthcare provider will form an initial assessment based on the subjective information provided by the patient. This initial assessment will then determine any observations (including tests) the healthcare provider makes during the encounter. The healthcare provider may then adjust the assessment in response to the observations. The treatment plan is then based on the final assessment.

Currently available electronic medical record systems push the healthcare provider to conduct the patient encounter by first entering observations, and then entering the assessment. Because this order does not comport with the manner in which a healthcare provider typically conducts a patient encounter, the currently available systems can be improved upon.

### BRIEF SUMMARY

We have provided an improved SOAP note system in which the order information is entered into the SOAP note is changed from the conventional order. In our system, initially, the patient enters relevant patient information regarding the reason for the particular visit into the system via a patient portal. This information will constitute the Subjective portion of the SOAP note. The Subjective information is stored in a record for the visit as part of the patient's overall medical record. The system provides a preliminary assessment based on the information provided by the patient and a suggested set of observations to be made by the practitioner. The practitioner retrieves the patient's electronic medical record and reviews the patient provided, subjective, information and the system provided initial assessment and suggested set of observations on a practitioner portal. The practitioner can modify the assessment by changing the diagnosis or adding one or more additional diagnoses to the assessment. Additionally, the practitioner can modify the suggested set of observations by deleting observations from and/or adding observations to the suggested set of observations. During the patient encounter, the practitioner will enter the observations made during the encounter. This is the Objective aspect of the SOAP note. Finally, based on the assessment and the objective information entered, the system will develop a proposed plan of action (the Plan of the SOAP note). The practitioner can add elements to, or remove elements from, the plan.

As seen, our new SOAP note system reverses the entry of the objective and assessment information. Thus, in our SOAP note system, the information is entered in the following order: Subjective-Assessment-Objective-Plan. Further, the SOAP note system provides proposed assessments, tests (objective information), and plans based on a condition reference. Our improved system allows information to be entered or produced in a clinically natural sequence of Subjective-Assessment-Objective-Plan (“SAOP”), rather than in the traditional SOAP sequence.

Additionally, for subsequent encounters with the same patient, the system will draw on prior SOAP notes to generate initial information for the subsequent encounter regarding the initial complaint/issue. By reverting to prior SOAP notes, the system will enhance the treatment of the patient by better ensuring that the practitioner has all the relevant information for the subsequent encounters.

In accordance with one aspect of the system, the system comprises a reference data structure comprising machine-readable relational links that associate diagnoses with corresponding care-plan elements, clinical tests, therapeutic modalities, and exercise prescriptions. In accordance with this aspect, the system auto-populates at least the objective and plan sections of the SOAP note based on the reference data structure.

In accordance with another aspect of the system, the system can, instead of a data base structure, comprise a dedicated large-language-model (“LLM”) inference engine that has been fine-tuned on de-identified medical literature, billing guidelines, and historical encounter data. At run-time the LLM generates (i) dynamically branching patient questionnaires, (ii) preliminary differential diagnoses with confidence scores, (iii) context-specific suggestions for objective examinations, and (iv) draft treatment plans, all of which are presented to—and may be edited by—the practitioner in real time. The tight coupling of the LLM with the patient's longitudinal record materially increases documentation accuracy, decreases encounter time, and ensures continuity of care across multiple visits.

In accordance with an aspect, the SOAP note system is part of an electronic medical record system comprising a patient portal, a practitioner portal, a server, and a non-volatile memory. The patient portal and practitioner portal are both in communication with the server and are adapted to send information to, and receiving information from, the server. An electronic medical record (EMR) data structure and a condition reference data structure are stored in the non-volatile memory. The condition reference data structure comprises data regarding diagnoses, care plans, treatment modalities, exercises, and tests. The system including instructions stored in the non-volatile memory for generating a SOAP note for a patient encounter related to a patient complaint. The instructions includes instructions for:
a) determining if the patient encounter is a first patient encounter or a subsequent patient encounter for the patient complaint;
b) generating a patient questionnaire and displaying the questionnaire on the patient portal; whereby:
   b1) for an initial visit for a patient complaint, the system includes instructions for providing the patient with an image of a body and enabling the patient to indicate which portion of the body is involved in the patient complaint, and generating further questions to the patient in response to answers received from the patient; and
   b2) for a second or subsequent visit for the same complaint, the system includes instructions for generating questions for patient as a function of information from a prior SOAP note for the patient complaint, wherein the questions generated elicit information from the patient regarding the status of the patient complaint;
c) receiving responses to questions in the questionnaire entered by a patient;
d) populating the subjective portion of the SOAP note with the patient responses to the questionnaire;
e) upon request, displaying the subjective portion of the SOAP note on the practitioner portal;
f) receiving a diagnosis from the practitioner and entering the diagnosis in the assessment portion of the SOAP note;
g) auto-populating a care plan in the assessment portion based on the diagnosis and care plan data in the condition reference data structure;
h) auto-populating the objective portion of the SOAP note with proposed tests for the practitioner to run and displaying the proposed tests on the practitioner portal, the proposed tests being selected based on the diagnosis and care plan data in the condition reference data structure;
i) receiving from the practitioner portal test result information entered into the practitioner portal by the practitioner for tests run and storing test data in the objective portion of the SOAP note; the test data comprising tests run and test results for the tests run; the tests run including at least a portion of the tests proposed by the system;
j) auto-populating the plan portion of the SOAP note with a proposed plan of treatment for the patient and displaying the proposed plan of treatment on the practitioner portal; the proposed plan of treatment being based on the diagnosis, the test results, and care plan data in the condition reference data structure;
k) receiving from the practitioner portal information regarding a prescribed treatment plan and storing the prescribed treatment plan in the plan portion of the SOAP note; the prescribed treatment plan comprising at least in part aspects of the proposed treatment plan;
l) generating a completed SOAP note from the information stored in the subjective portion, assessment portion, objective portion, and plan portion of the SOAP note and displaying the completed SOAP note on the practitioner portal;
m) enabling the practitioner to electronically sign the completed SOAP note;
n) upon receipt of an electronic signature for the SOAP note, storing the SOAP note as a SOAP note record for the particular patient encounter in an electronic medical record for the patient.

In accordance with an aspect of the system, the system being adapted to (1) determine if a vital statistic (such as blood pressure, weight, BMI, pulse, etc.) is abnormal, generate a warning regarding the abnormal vital statistic, and display the warning on all screens of an electronic medical record for the patient; or (2) enable the provider to enter an alert regarding a condition of the patient and display the alert on all screens of the electronic medical record for the patient.

In accordance with an aspect of the system, the server can be remote or local and the non-volatile memory can be remote or local.

In accordance with an aspect of the system, the patient portal and practitioner portal can each comprise one or more of kiosks, laptop computers, tablet computers, cell phones, or combinations thereof.

In accordance with an aspect of the system, the system is adapted to enable the practitioner to request additional tests, in which case, the test data wo; include the additional tests and results of the additional tests.

In accordance with an aspect of the system, the proposed plan of treatment comprises one or more of proposed imaging and/or tests, proposed treatments, proposed modalities, proposed supplements and/or supplies, and proposed exercises.

In accordance with an aspect of the system, the system is adapted to enable the practitioner to prescribe additional imaging and/or tests, treatments, modalities, supplements and/or supplies, and/or exercises not proposed by the system; wherein the prescribed plan of action comprises at least a portion of the proposed imaging and/or tests, proposed treatments, proposed modalities, proposed supplements and/or supplies, and proposed exercises and the additional prescribed imaging and/or tests, treatments, modalities, supplements and/or supplies, and exercises.

In accordance with an aspect of the system, upon completion of the objective portion of the SOAP note, test data comprising tests run and test results is synced to, or stored in, a test panel of the EMR which contains a history of tests run on the patient.

In accordance with an aspect of the system, where upon completion of the plan portion of the SOAP note, treatment plan data comprising the prescribed treatment plan is synced to, or stored in, a treatment panel of the EMR.

In accordance with an aspect of the system, the treatment panel comprises (i) a modalities panel comprising data regarding prescribed modalities and (ii), an exercise panel comprising data regarding prescribed tests.

In accordance with an aspect of the system, the system includes means for (1) indicating when modalities and/or exercises have been completed and (2) means for tagging the modalities and/or exercises completed in-office with a billing indicator; whereby the system is adapted to provide the billing indicator to a billing system which can generate a claim to be submitted to an insurance company and/or an invoice to be provided to the patient.

In accordance with another aspect of the SOAP note system, the electronic medical record system comprises a patient portal, a practitioner portal, a server, and a non-volatile memory in which is stored an electronic medical record (EMR) data structure and a condition reference data structure, the condition reference data structure comprising data regarding diagnoses, care plans, treatment modalities, exercises, and tests; the patient portal and practitioner portal both being in communication with the server and being adapted to send information to, and receiving information from, the server. The system includes instructions stored in the non-volatile memory for generating a SOAP note comprising a subjective portion, assessment portion, objective portion, and plan portion. The method includes:
a) determining if the patient encounter is a first patient encounter or a subsequent patient encounter for the patient complaint;
b) generating a patient questionnaire and displaying the questionnaire on the patient portal; the step of generating the patient questionnaire comprising:
   b1) for an initial visit for a patient complaint, providing the patient with an image of a body and enabling the patient to indicate which portion of the body is involved in the patient complaint, and generating further questions to the patient in response to answers received from the patient; and
   b2) for a second or subsequent visit for the same complaint, generating questions for patient as a function of information from a prior SOAP note for the patient complaint, wherein the questions generated illicit information from the patient regarding the status of the patient complaint;
c) receiving responses from the patient portal to questions in the questionnaire entered by a patient;
d) populating the subjective portion of the SOAP note with the patient responses to the questionnaire;
e) upon request, displaying the subjective portion of the SOAP note on the practitioner portal;
f) receiving a diagnosis from the practitioner and entering the diagnosis in the assessment portion of the SOAP note;
g) auto-populating a care plan in the assessment portal based on the diagnosis and care plan data in the condition reference data structure;
h) auto-populating the objective portion of the SOAP note with proposed tests for the practitioner to run and displaying the proposed tests on the practitioner portal, the proposed tests being selected based on the diagnosis and care plan data in the condition reference data structure;
i) receiving from the practitioner portal test result information entered into the practitioner portal by the practitioner for tests run and storing test data in the objective portion of the SOAP note; the test data comprising tests run and test results for the tests run; the tests run including at least a portion of the tests proposed by the system;
j) auto-populating the plan portion of the SOAP note with a proposed plan of treatment for the patient and displaying the proposed plan of treatment on the practitioner portal; the proposed plan of treatment being based on the diagnosis, the test results, and care plan data in the condition reference data structure;
k) receiving from the practitioner portal information regarding a prescribed treatment plan and storing the prescribed treatment plan in the plan portion of the SOAP note; the prescribed treatment plan comprising at least in part aspects of the proposed treatment plan;
l) generating a completed SOAP note from the information stored int eh subjective portion, assessment portion, objective portion, and plan portion of the SOAP note and displaying the completed SOAP note on the practitioner portal;
m) enabling the practitioner to electronically sign the completed SOAP note;
n) upon receipt of an electronic signature for the SOAP note, storing the SOAP note as a SOAP note record for the particular patient encounter in an electronic medical record for the patient.

In accordance with an aspect of the method, the method including steps of: (1) determining if a vital statistic (such as blood pressure, weight, BMI, pulse, etc.) is abnormal, generating a warning regarding the abnormal vital statistic, and displaying the warning on all screens of an electronic medical record for the patient; or (2) enabling the provider to enter an alert regarding a condition of the patient and displaying the warning on all screens of the electronic medical record for the patient.

In accordance with an aspect of the method, wherein the method includes a step of enabling the practitioner to request additional tests; wherein the test data includes the additional tests and results of the additional tests.

In accordance with an aspect of the method, the proposed plan of treatment comprises one or more of proposed imaging and/or tests, proposed treatments, proposed modalities, proposed supplements and/or supplies, and proposed exercises; wherein the method includes a step of enabling the practitioner to prescribe additional imaging and/or tests, treatments, modalities, supplements and/or supplies, and/or exercises not proposed by the system; wherein the prescribed plan of action comprises at least a portion of the proposed imaging and/or tests, proposed treatments, proposed modalities, proposed supplements and/or supplies, and proposed exercises and the additional prescribed imaging and/or tests, treatments, modalities, supplements and/or supplies, and exercises.

In accordance with an aspect of the method, the method includes a step of, upon completion of the objective portion of the SOAP note, syncing or storing test data comprising tests run and test results, to or in a test panel containing a history of tests run on the patient.

In accordance with an aspect of the method, upon completion of the plan portion of the SOAP note, the method includes a step of syncing or storing treatment plan data comprising the prescribed treatment plan to or in a treatment panel.

In accordance with an aspect of the method, the treatment panel comprises (i) a modalities panel comprising data regarding prescribed modalities and (ii), an exercise panel comprising data regarding prescribed tests.

In accordance with an aspect of the method, the method includes steps of (1) indicating when modalities and/or exercises have been completed and (2) tagging the modalities and/or exercises completed in-office with a billing indicator; whereby the system is adapted to provide the billing indicator to a billing system which can generate a claim to be submitted to an insurance company and/or an invoice to be provided to the patient.

In a further aspect, a computer-implemented electronic medical record system for automatically generating, storing, and securing a SOAP note for a patient encounter is provided wherein, the system comprises:
a. a server comprising at least one processor and a non-transitory computer-readable medium;
b. a patient portal and a practitioner portal, each being adapted to communicate with the server to exchange data with the server over a communication network;
c. the non-transitory computer-readable medium storing:
   (i) an electronic medical record (EMR) data structure that, for each patient, includes at least one encounter record, each encounter record having at least a subjective portion, an assessment portion, an objective portion, and a plan; and
   (ii) a condition reference data structure comprising machine-readable relational links that associate diagnoses with corresponding care-plan elements, clinical tests, therapeutic modalities, and exercise prescriptions; and
d. executable instructions which, when executed by the processor, cause the server to:
   (1) prior to a first encounter for a particular patient complaint, transmit to the patient portal a dynamically generated electronic questionnaire that is iteratively built in real time in response to patient interaction with an anatomical graphical user interface, the questionnaire being restricted to questions pertinent to a body region selected by the patient;
   (2) receive patient responses from the patient portal and automatically store the responses in the subjective portion of a newly created encounter record within the EMR data structure;
   (3) render for display on the practitioner portal the stored subjective portion together with provisional assessment data items automatically retrieved from the condition reference data structure by matching the patient responses to linked diagnoses;
   (4) receive final assessment data via the practitioner portal and store the final assessment data in the assessment portion of the encounter record, the final assessment data comprising the provisional assessment data if approved by the practitioner or practitioner-edited assessment data;
   (5) automatically compile, based on the final assessment data and the relational links in the condition reference data structure, a set of proposed objective clinical tests ordered according to diagnostic utility, transmit the set to the practitioner portal, receive entered test results, and store the results in the objective portion of the encounter record;
   (6) automatically generate, using the stored assessment data and objective test results, an individualized treatment plan comprising one or more of an imaging order, a therapeutic modality, and an exercise prescription, the treatment plan being derived by traversing the relational links of the condition reference data structure, and store the treatment plan in the plan portion of the encounter record;
   (7) generate, within the EMR data structure and without user transcription, a structured electronic SOAP note that orders the stored subjective, assessment, objective, and plan portions sequentially as Subjective-Assessment-Objective-Plan;
   (8) display the generated SOAP note on the practitioner portal for electronic signature; and
   (9) for each subsequent encounter relating to the same patient complaint, automatically initiate a new encounter record that imports, as default values, data from at least the assessment and plan portions of a most recent prior encounter record, repeats operations (1)-(8) using updated questionnaire responses, and thereby minimizes redundant data entry across encounters.

In accordance with a further aspect of the system, a computer-implemented electronic medical record system for automatically generating, storing, and securing a SOAP note for a patient encounter is provided wherein, the system comprises:
a. a server comprising at least one processor and a non-transitory computer-readable medium;
b. a patient portal and a practitioner portal, each being adapted to communicate with the server to exchange data with the server over a communication network;
c. the non-transitory computer-readable medium storing:
   (i) an electronic medical record (EMR) data structure that, for each patient, includes at least one encounter record, each encounter record having at least a subjective portion, an assessment portion, an objective portion, and a plan portion; and
   (ii) a large language model (LLM) trained on medical data and capable of associating diagnoses with corresponding care-plan elements, clinical tests, therapeutic modalities, and exercise prescriptions; and
d. executable instructions which, when executed by the processor, cause the server to:
   (1) prior to a first encounter for a particular patient complaint, transmit to the patient portal a dynamically generated electronic questionnaire that is iteratively built in real time in response to patient interaction with an anatomical graphical user interface, the questionnaire being restricted to questions pertinent to a body region selected by the patient;
   (2) receive patient responses from the patient portal and automatically store the responses in the subjective portion of a newly created encounter record within the EMR data structure;
   (3) render for display on the practitioner portal the stored subjective portion together with provisional assessment data items automatically generated by the large language model (LLM) by analyzing the patient responses and suggesting linked diagnoses;
   (4) receive practitioner-edited assessment data via the practitioner portal and store the edited assessment data in the assessment portion of the encounter record;
   (5) automatically compile, based on the edited assessment data and the output of the large language model (LLM), a set of proposed objective clinical tests ordered according to diagnostic utility, transmit the set to the practitioner portal, receive entered test results, and store the results in the objective portion of the encounter record;
   (6) automatically generate, using the stored assessment data and objective test results, an individualized treatment plan comprising one or more of an imaging order, a therapeutic modality, and an exercise prescription, the treatment plan being derived by querying the large language model (LLM), and store the treatment plan in the plan portion of the encounter record;
   (7) generate, within the EMR data structure and without user transcription, a structured electronic SOAP note that orders the stored subjective, assessment, objective, and plan portions sequentially as Subjective-Assessment-Objective-Plan;
   (8) display the generated SOAP note on the practitioner portal for electronic signature, and, upon receipt of the signature, cryptographically lock the encounter record against further alteration; and
   (9) for each subsequent encounter relating to the same patient complaint, automatically initiate a new encounter record that imports, as default values, data from at least the assessment and plan portions of a most recent prior encounter record, repeats operations (1)-(8) using updated questionnaire responses, and thereby minimizes redundant data entry across encounters.

### BRIEF DESCRIPTION OF THE DRAWINGS

FIG. 1 is block diagram of the components of an electronic health record/SOAP note system;

FIG. 2A and 2B are diagrams of the flow of information through the system to generate a SOAP note;

FIG. 2C is a block diagram of a patient's electronic health record, showing two encounters for a single issue;

FIG. 3 is an illustrative screen shot of a patient questionnaire displaying a diagram of a body;

FIGS. 4-6 are illustrative screen shots of the patient questionnaire enabling a patient to enter information regarding the patient's condition;

FIG. 7 is a first screen of a patient electronic medical record (EMR) of the system showing general patient information;

FIG. 8 is a screen shot of an encounter detail screen of the EMR showing the Subjective information portion of a patient electronic medical record for an encounter or visit;

FIGS. 9A-B are screen shots of the encounter detail screen showing an Assessment information portion of a patient electronic medical record for an encounter or visit;

FIG. 10 is a screen shot of the encounter detail screen showing an Objective information portion of a patient electronic medical record for an encounter or visit;

FIGS. 10A-G show the joint, range of motion, myofascial, orthopedic,

and neurological panels of the Objective information portion of the EMR;

FIG. 11A-B is an illustrative screen shot of the encounter detail

screen showing a plan portion of a patient electronic medical record for an encounter or visit;

FIGS. 12A-F are “order test”, “treatment detail”, “add modality”, “supplement & supplies”, and “exercise” pop-up windows accessed form the “plan” portion of the encounter detail screen;

FIG. 13 is a view of a completed plan portion of the patient electronic medical record for the particular patient visit;

FIG. 14 is a view of the “modalities” tab of the patient EMR;

FIG. 15 is a view of the “exercises” tab of the patient EMR;

FIG. 16 is a view of the “test” tab of the patient EMR;

FIG. 17 is a view of the “vitals” tab of the patient EMR;

FIG. 18 is a view of the “communications” tab of the patient EMR;

FIGS. 19 and 19A-C show a completed SOAP note, as prepared by the system;

FIG. 19D shows the bottom portion of a signed SOAP note;

FIG. 20 shows a “chart” tab of the patient EMR;

FIGS. 21A and B show a “memo” tab of the EMR and a pop-up window to add a memo to the memo tab;

FIG. 22 shows an “encounter” tab of the EMR; and

FIG. 23-26 shows the subjective, assessment, objective, and plan portions, respectively of a SOAP note for a subsequent visit by the patient.

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

Although the electric medical record system is described with reference to a chiropractic setting, it will be apparent that the system can be adapted for use in any type of health-related practice, such as a medical (internal, allergy, geriatric, etc.) practice, surgical practice, dental practice, psychiatric practice, physical therapy practice, etc.

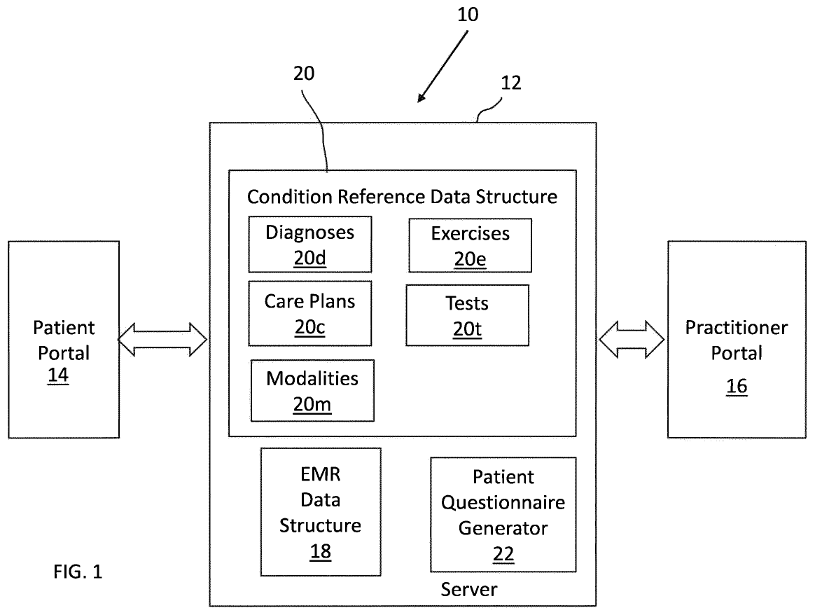

Turning initially to FIG. 1, the system 10 comprises a server 12, a patient portal 14, and a practitioner portal 16. The patient and practitioner portals are both computer-type devices. They can be kiosks, lap top computers, tablet computers, cell phones, etc. Further, the patent and practitioner portals can be web enabled to allow for respective information to be entered, edited, or reviewed remote from the practitioner office. The patient and practitioner portals 14, 16 thus include at least a processor, a display screen and data entry means (such as a virtual or physical keyboard, touch screen, etc.). The system 10 can include multiple patient and practitioner portals 14, 16 to enable multiple patients and practitioners to engage with the system concurrently.

The server 12 similarly includes a processor and storage means on which is stored an electronic medical record (EMR) data structure 18, a condition reference 20, and a patient questionnaire generator 22. The EMR data structure 18 and condition reference 20 can be stored locally or can be stored in remote storage (i.e., “in the cloud”). The server 12 is adapted to communication with the patient portal 14 and practitioner portal 16, and similarly, the patient portal 14 and practitioner portal 16 are adapted to communicate with the server. The server can be a local server, in which case, the communication between the server 12 and the portals 14, 16 can be hard wired (such as over ethernet cables) or over-the-air (such as via a Wi-Fi connection). Alternatively, the server can be a remote server, in which case, the communication between the portals 14, 16 and the server 12 will be over the air (such as through the internet or via Bluetooth technology). Any desired communication technology can be used.

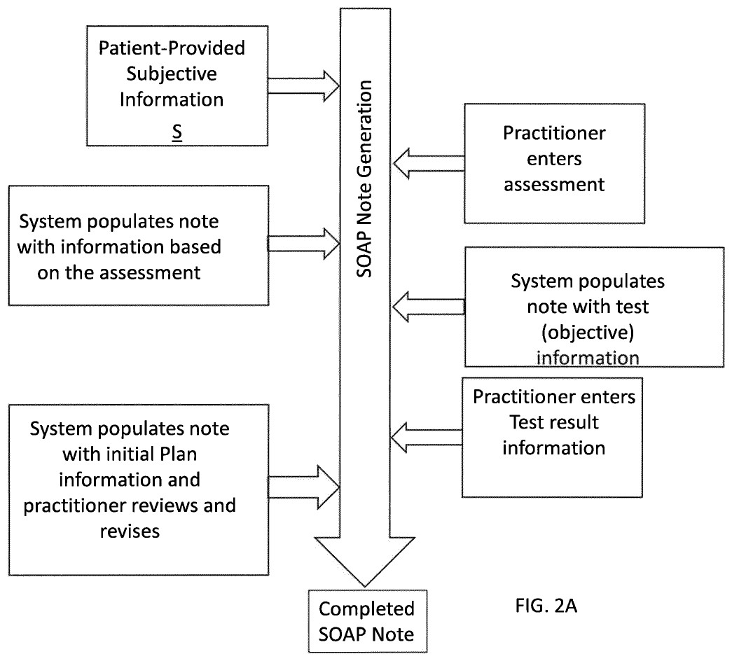

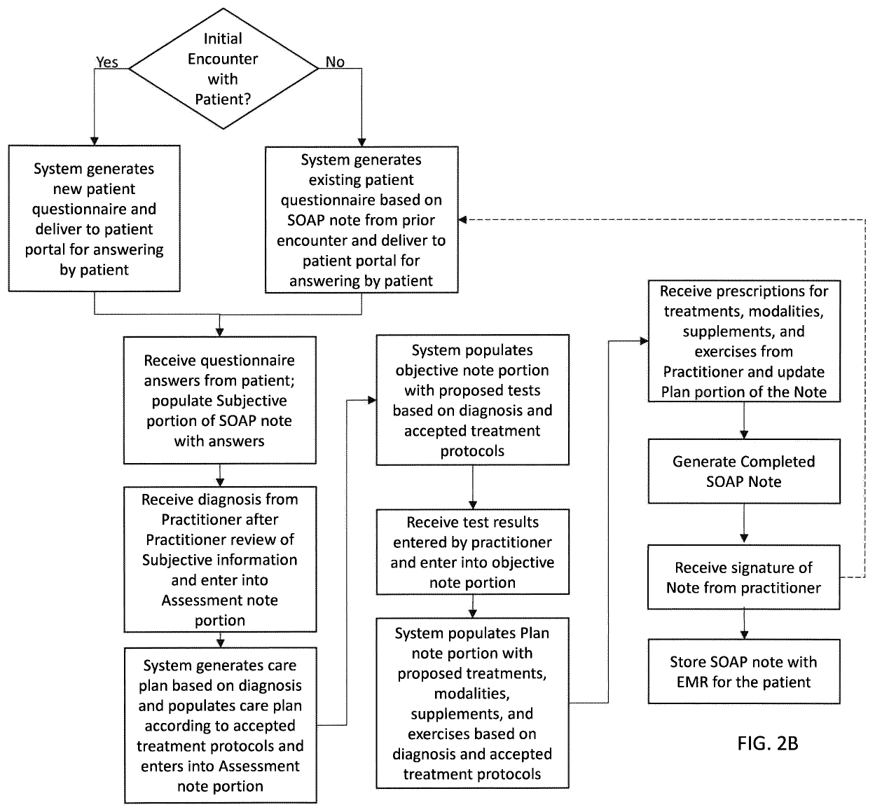

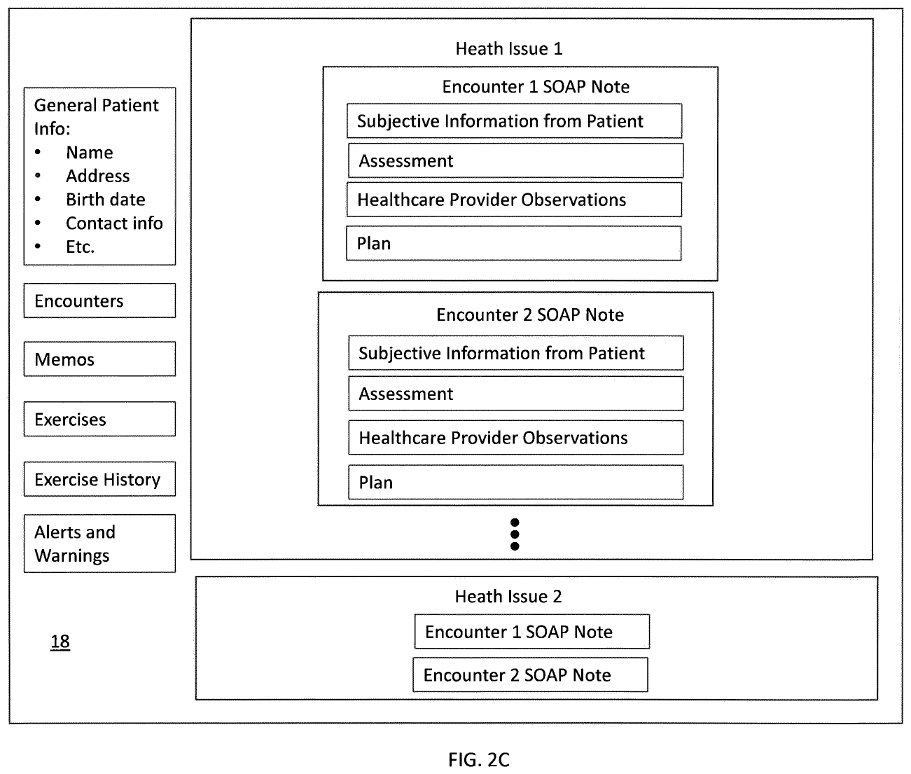

The EMR data structure 18 stores the longitudinal electronic health records for each patient. A schematic record of the EMR data structure is shown in FIG. 2C. As seen, the record includes general patient information, as wells as the SOAP note for each patent encounter for each health issue.

The condition reference 20 contains data relating to the treatment of conditions. In the example of a chiropractic practice, the data structure contains information relating to the treatment of musculoskeletal disorders which are treated by chiropractors. To this end, the condition reference 20 includes data or information relating to diagnoses 20d, modalities 20m, exercises 20e, tests 20t, and care plans 20c that are typically performed or prescribed for specific conditions. As will be discussed further below, the condition reference 20 is used to initially auto-populate the various portions of the SOAP note, which can then be edited by the practitioner.

In one version, the condition reference 20 can be a data structure that is analogous to a complex relational database, in which diagnoses, modalities, exercises, tests, and care plans are tied together, thereby enabling the system to propose assessments, tests and care plans based, initially, on the subjective information entered by the patient. In this version, the data structure comprises machine-readable relational links that associate diagnoses with corresponding care-plan elements, clinical tests, therapeutic modalities, and exercise prescriptions.

In another version, the condition reference can comprise a dedicated large-language-model (“LLM”) inference engine that has been fine-tuned, for example, on de-identified medical literature and other public domain biomedical literature, standardized coding manuals (e.g., ICD-10-CM, CPT, HCPCS), billing guidelines, and anonymized historical encounter data provided by the practitioner. When run the LLM generates (i) dynamically branching patient questionnaires, (ii) preliminary differential diagnoses with confidence scores, (iii) context-specific suggestions for objective examinations, and (iv) draft treatment plans, all of which are presented to, and may be edited by, the practitioner in real time. The tight coupling of the LLM with the patient's longitudinal record materially increases documentation accuracy, decreases encounter time, and ensures continuity of care across multiple visits. By using practitioner provided historical encounter data, the AI system would be able to “learn” the practitioner's preferred diagnoses, tests, and treatments, and suggest these preferred diagnoses, tests, and treatments as the SOAP note is generated.

Lastly, the patient questionnaire generator 22 generates a questionnaire to be completed by the patient prior to each meeting with the practitioner. The patient questionnaire generator will build the questionnaire presented to the patient as the patient answers questions. For example, if the patient complains of neck issues, the questionnaire generated by the patient questionnaire generator will be relevant to the neck issue. This enable the system to present the patient with a questionnaire the is directed to the patient's specific issue. This thus avoids the use of a general questionnaire which may ask the patient for superfluous information.

In operation, as a first step, the patient will access the system 12 via the patient portal 14 to fill out a questionnaire which is delivered by the server to the patient portal to enable the patient to fill in and complete the questionnaire. This questionnaire will enable the patient to provide information regarding the reason for the particular visit/encounter. The questionnaire, as just noted, is generated by the patient questionnaire generator 22.

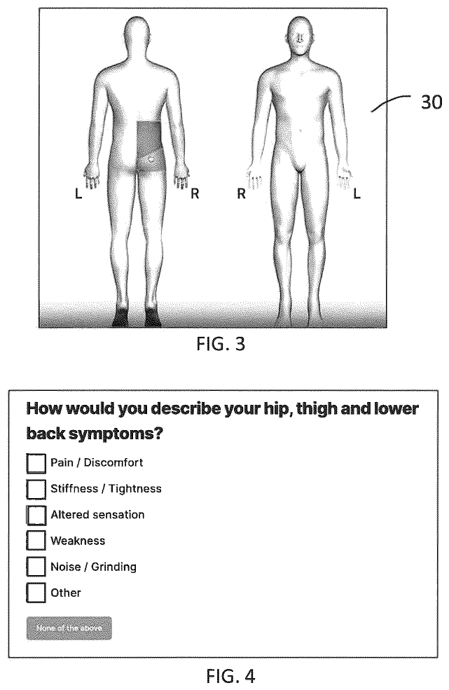

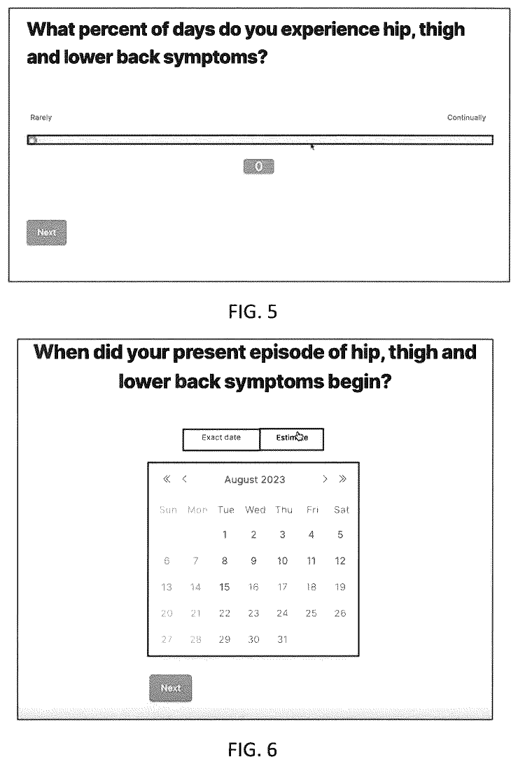

In a chiropractic setting the questionnaire generator will initially display a diagram 30 of a body (FIG. 3) on the patient portal. As seen in FIG. 3, the diagram shows the front and back of a body. Using a mouse, stylus, or touch screen, the patient can indicate the portion of the body affected. In FIG. 3, the lower back and buttock on the patient's right side has been selected. The questionnaire generator uses this input from the patient to build the questionnaire in real time. The system can, if desired, prevent the patient from combining unrelated issues. For example, in FIG. 3, the patient illustratively indicated that the symptoms involved the lower back and hip. If the system is set to prevent the patient from entering unrelated systems, the system could, for example, prevent the patient from also noting a wrist issue (which would unlikely be related to the lower back and hip issue). The questionnaire, using this input, presents the patient with the questions shown in FIGS. 5-6 to gather general information regarding the specific condition, such as how frequently the symptoms are experienced and when did they begin. The questionnaire generator 22 can then present the patient with a series of more specific questions regarding the specific symptoms (pain, weakness, mobility, etc.), the intensity of any pain, thoughts as to what caused the symptoms (repetitive activity, accident, employment-related, etc.), whether the symptoms have improved or gotten worse since their onset, what affects the symptoms, what relieves the symptoms, etc. These questions can be answered via radio buttons or check boxes as shown in FIG. 4 or via sliding scale as shown in FIG. 5. A calendar as shown in FIG. 6 can be provided to select date information (such as symptom onset).

The questionnaire generator builds the questionnaire in real time as the patient answers the questions. In one version, the questions provided to the patient by the questionnaire generator can be based on a question tree. In an Al-version, the questionnaire generator 22 can operate as a thin orchestration layer that streams conversational context to the LLM engine. On receiving an encounter request, the generator constructs an initial prompt that includes the body-map token identifying the anatomic region(s) selected by the patient and any relevant carry-over notes from prior encounters. The LLM then returns an ordered list of follow-up questions whose branching logic is determined dynamically from patient responses. Because the question sequence is authored on the fly by the model, the system avoids static decision trees and eliminates redundant or irrelevant inquiries.

For a chiropractic practice, a portion of the questions are related to the Oswestry Disability Index. As can be appreciated, the questions asked of the patient will vary depending on the symptoms indicated in initial screens of the questionnaire. For example, if the patient indicates the issue is pain or discomfort, the patient can be presented with a question regarding the type of pain (i.e., sore, dull, ache, shooting, burning, throbbing, etc.) which can be part of the condition reference or which can be an independent data structure of the system. Finally, the patient can be provided with a box (not shown) to enter free form text to let the provider know any additional information which the patient may believe is relevant. The patient provided information is become the subjective information S in the SOAP note, as shown in FIG. 2. Because the questionnaire generator 22 builds the questionnaire in real time as the patient answers the questions, the subjective information provided by the patient (and which will be presented to the practitioner) will not contain extraneous information that might be provided if the patient were presented with a general questionnaire.

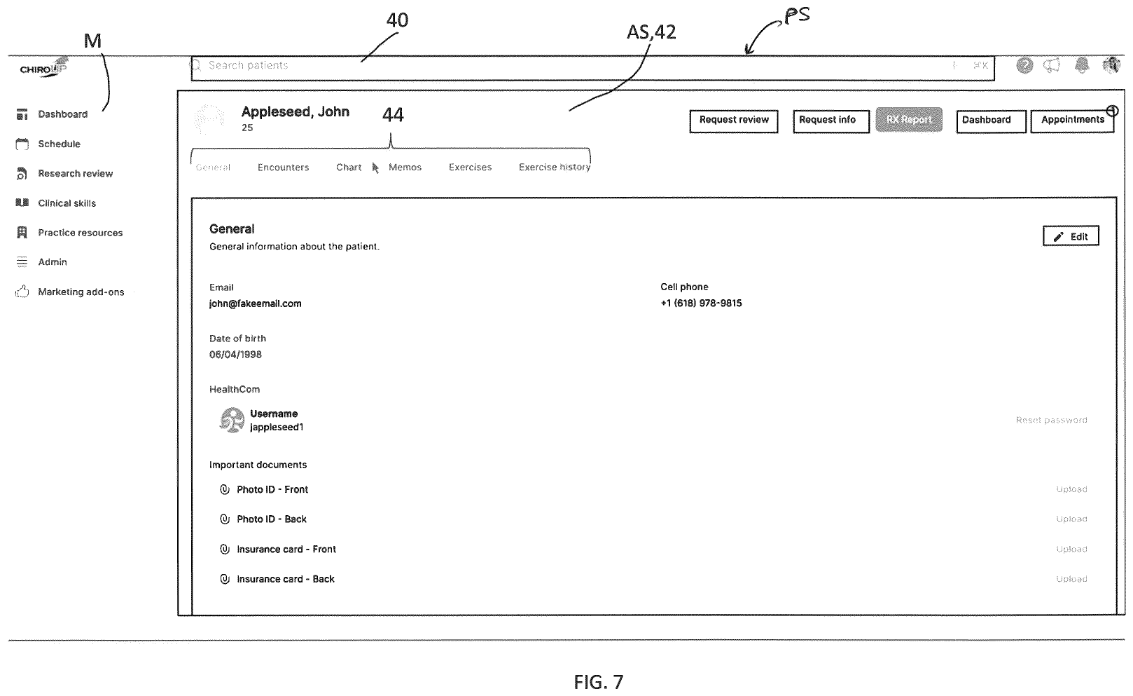

FIG. 7 shows a practitioner screen PS of the practitioner portal. The practitioner screen PS includes an active screen AS and a main menu M to the left of the active screen AS. Additionally, a patient search bar 40 can be positioned above the active screen AS. The main menu M and patient search bar will always be present regardless of the information shown in the active screen AS. The main menu M includes selections such as “Dashboard” which will allow the practitioner to select various functions of the system (such as billing), “schedule” which when selected displays a calendar in the active screen showing scheduled patient visits for a selected period (i.e., day, week, or month), “Research Review” which will allow the practitioner to access information regarding topics of interest, “Clinical Skills” which links to or is adapted to query the information in the condition reference 20, “Practice Resources” which provide access to information regarding, for example, exercises that might be prescribed for the patient, and “Admin” which provides for access to administrative actions, such as adding new users, and “Marketing add-ons”.

In FIG. 7, the initial screen of a patient's EMR 42 is shown in the active screen AS. This initial screen includes general information (i.e., name, birth date, contact information, etc.) regarding the patient. Below the patient's name, the EMR 42 includes an EMR menu 44 which enables the practitioner to access different pages or sections of the EMR. These include a general page (which is shown), an encounter page (to be described below), a chart page (which provides a history of a particular patient encounter), a memo page (which can include notes from the practitioner regarding the patient), an exercises page (which can include exercises prescribed for the patient), and an exercise history page (which can include additional information regarding patient exercise). The patent's EMR can be reached, for example, by clicking on the relevant appointment in the “schedule” module of the main menu M or by searching for the patient by name in the search bar 40.

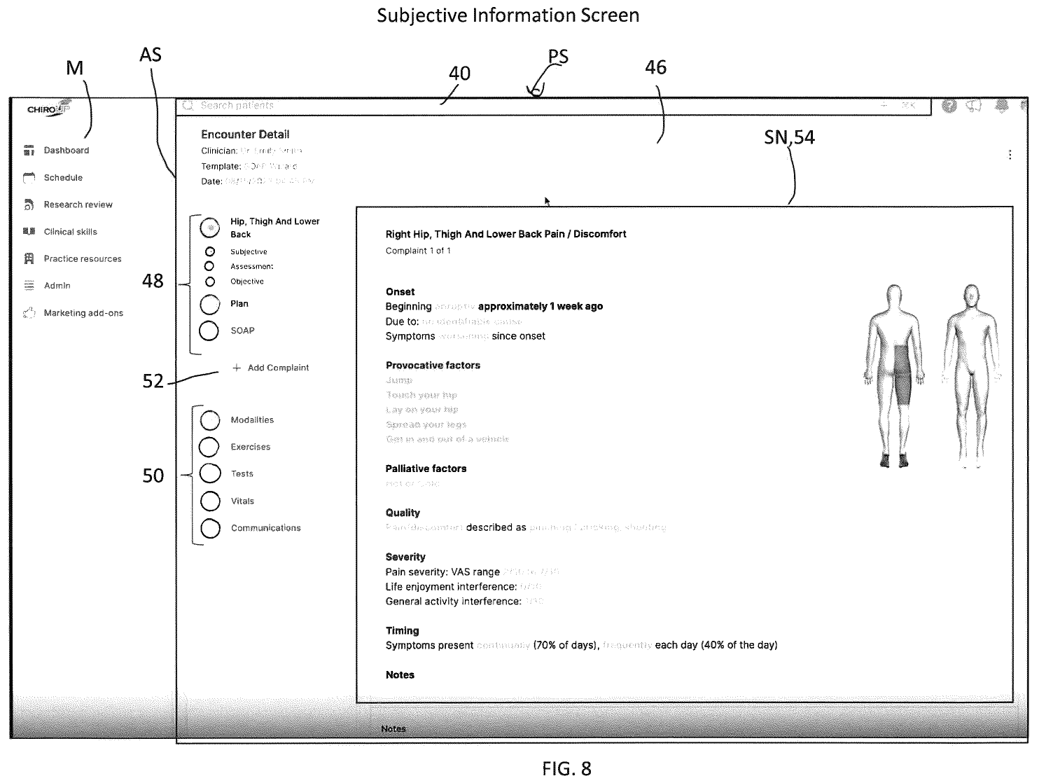

By clicking on the “encounters” tab in the EMR menu 44, the active screen will be replaced with an encounter detail screen 46 (FIG. 8). The system generates a new encounter detail record for each visit for the patient. The encounter detail screen includes a SOAP note window SN. To the left of the SOAP note window SN, the encounter screen 46 includes a SOAP note navigation menu 48 which will allow the practitioner to navigate through and populate the SOAP note as will become more apparent below. Below the SOAP note menu is a module menu 50 which enables the practitioner to navigate through “modalities”, “exercises”, “tests”, “visits” and “communications” modules of the EMR. As will become apparent below, these menus allow for the practitioner to add additional details to the patient's EMR.

In FIG. 8, the SOAP note section SN is shown displaying the subjective information section 54. The Subjective information section 54 is prepopulated with the information provided by the patient when answering the questionnaire. The Subjective information section 54 thus includes a brief title for the complaint, its onset, issues that may aggravate the condition, actions that may alleviate some of the pain, the type of pain, the severity of the pain, etc. In the illustrative image of FIG. 8, the information displayed relates to a patient experiencing hip and/or lower back pain or discomfort. The categories of information may change depending on the particular complaint. Because the subjective information is prepopulated, the practitioner can review the Subjective information prior to meeting with the patient, making the patient encounter more productive. If the subjective information is completed to the practitioner's satisfaction, the practitioner can select “Assessment” in the SOAP note menu 48. However, if there are additional conditions, the practitioner can click the “add complaint” button 52 to add additional conditions/complaints the patient may have. Additionally, the practitioner has the ability to edit the subjective information in response to the practitioner's discussion with the patient. Although the practitioner can edit the subjective information, the system will preserve a record of the patient's answers to the questionnaire, and thus will preserve the patient's initial subjective information. Lastly, although not shown, the subjective note section can include a notes box into which the practitioner can enter notes relevant to the subjective section. This notes section can be tagged such that it will automatically carry over to the subjective information section of a SOAP note for a next visit for this complaint. This will make it easier for the practitioner to be reminded of a particular detail which s/he may feel important to the ongoing treatment of the patient.

If for some reason, the patient was not able to answer the questionnaire prior to the appointment with the practitioner, the practitioner can manually generate a SOAP note for the patient. This, however, would require that the practitioner go through the questionnaire with the patient in order to complete the subjective portion of the SOAP note.

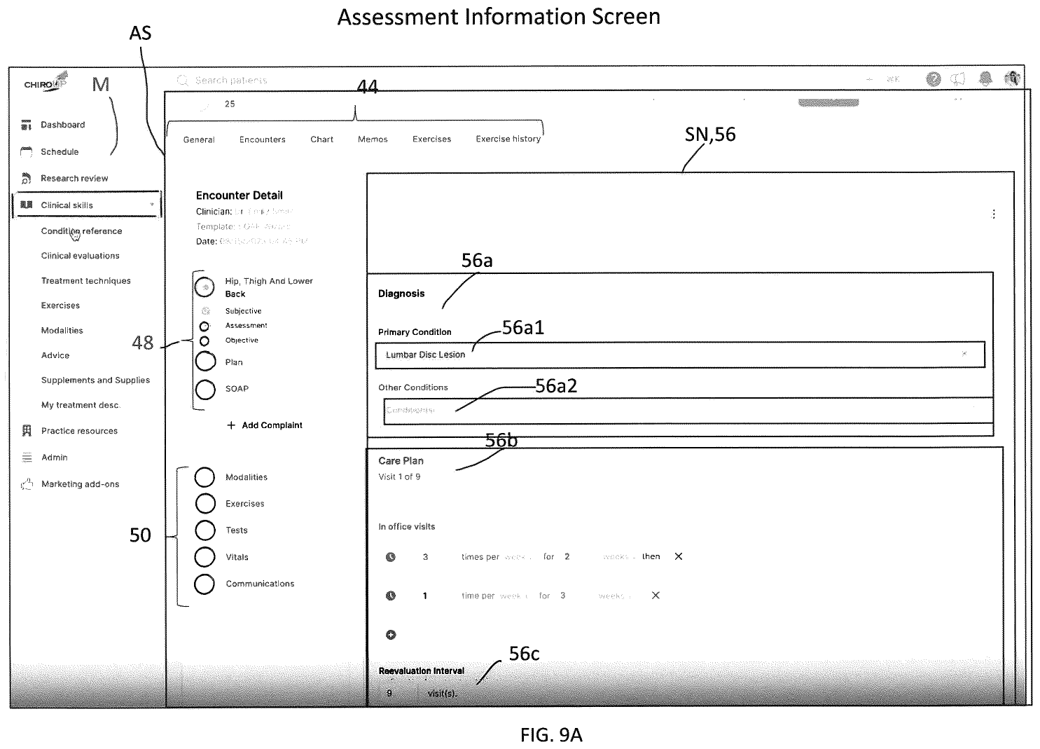

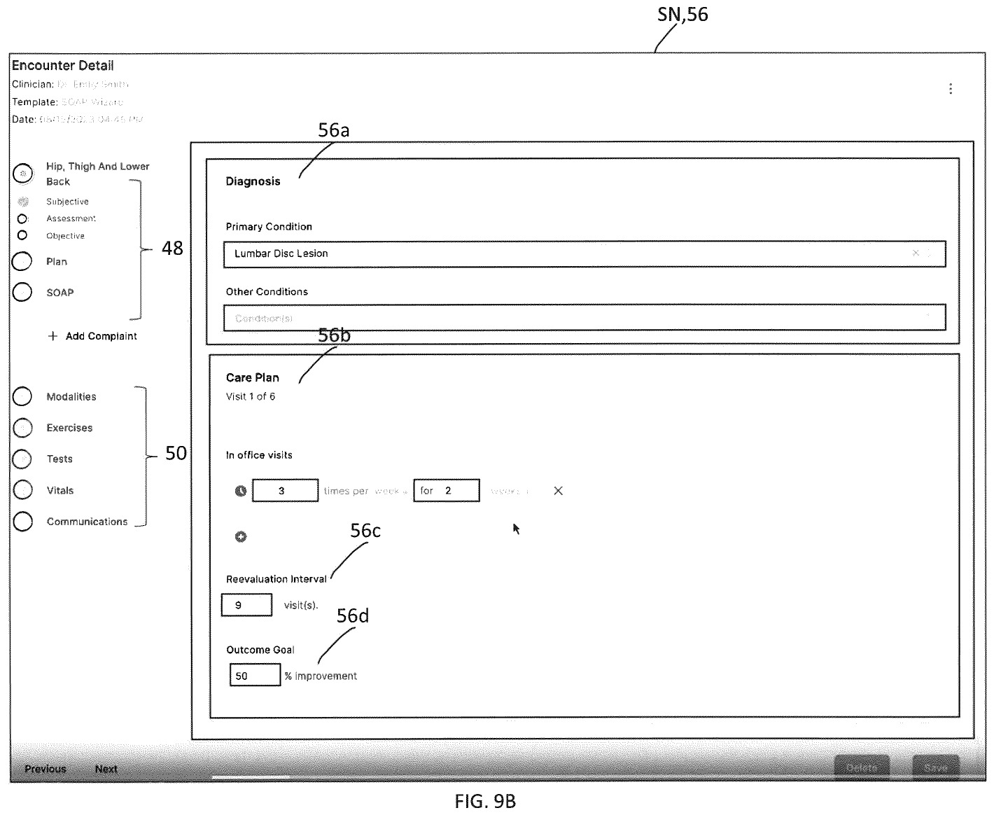

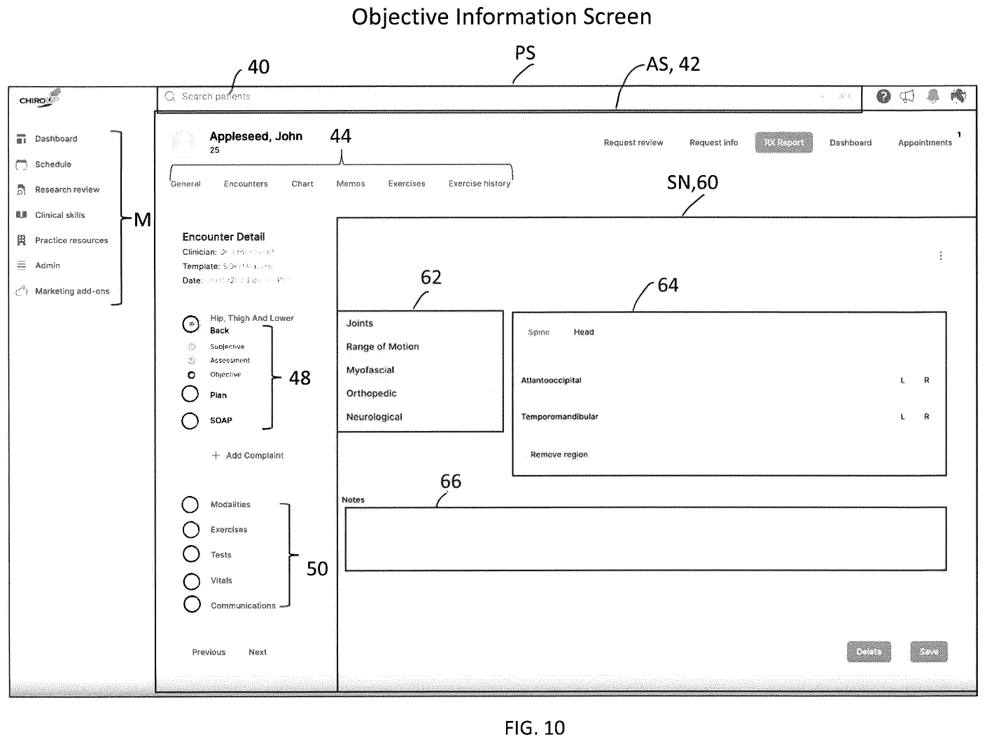

After clicking the “Assessment” selection on the SOAP note menu 48, the system will display an assessment section 56 in the SOAP note window SN, shown in FIGS. 9A,B. As seen, the main menu M, the SOAP note menu 48, and the module menu 58 continue to be displayed in the active screen AS. These menus will be shown in all the various screens of the encounter detail to enable the practitioner to navigate through the encounter detail and the system itself. The assessment section 56 includes a diagnosis section 56a, a care plan section 56b, a reevaluation interval section 56c, and an outcome goal section 56d.

To the left of the active screen AS, the “clinical skills” portion of the main menu M is shown expanded. This can be selected, or it can automatically expand upon opening of the assessment window. The “clinical skills” section, as noted above, links to, or is adapted to query, the condition reference 20 and provides information that will be used by the system to auto-populate a large portion of the SOAP note. To this end, the Clinical Skills section includes information relating to condition reference, clinical evaluations, treatment techniques, exercises, modalities, advice, and supplements and supplies. The information that can be retrieved from these clinical skills will be based on the diagnosis entered.

In our system, we have moved the “assessment” portion of the SOAP note to be before the “objective” section of the note because the practitioner, based upon his/her training and experience, will have an initial assessment of the patient's condition. This assessment will then determine the information the practitioner will gather during the patient visit (i.e., the objective information) which will then be used to develop a treatment plan. Thus, the practitioner enters his/her initial assessment into the diagnosis section 56a of the assessment screen 56. As seen, the practitioner can enter a primary condition in box 56a1, and if warranted, can enter in additional conditions or diagnoses in box 56a2 which the practitioner believes to be relevant to the patient's complaint. The conditions or diagnosis can be entered from a pop-up screen which allows the practitioner to more easily select conditions or diagnosis. The conditions/diagnosis in this pop-up screen can be organized by area of the body, enabling the practitioner to more quickly hone in on the proper diagnosis. Further, the system can, based on the information from the subjective section, highlight or otherwise suggest likely diagnoses to the practitioner. The practitioner can accept or reject such suggested diagnoses. Once a diagnosis is selected, the practitioner can select ICD (International Classification of Deases) codes for the particular diagnoses. More than one code per condition can be selected if necessary.

Upon entry of the diagnosis by the practitioner, the system will then auto-populate a majority of the SOAP note based upon standard treatment techniques. This information is contained in the condition reference. Thus, in the assessment window 56, the system will auto-populate the care plan section 56b, reevaluation interval sections 56c, and outcome goal 56d based on the standard of care. The practitioner can override, edit or alter the care plan, reevaluation interval, and outcome goal sections based on the practitioner's experience.

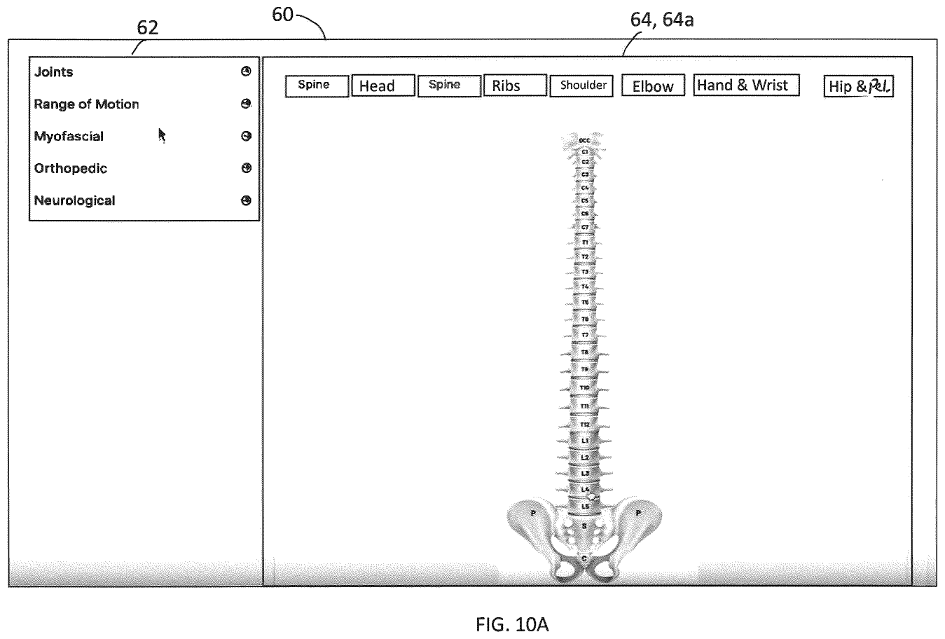

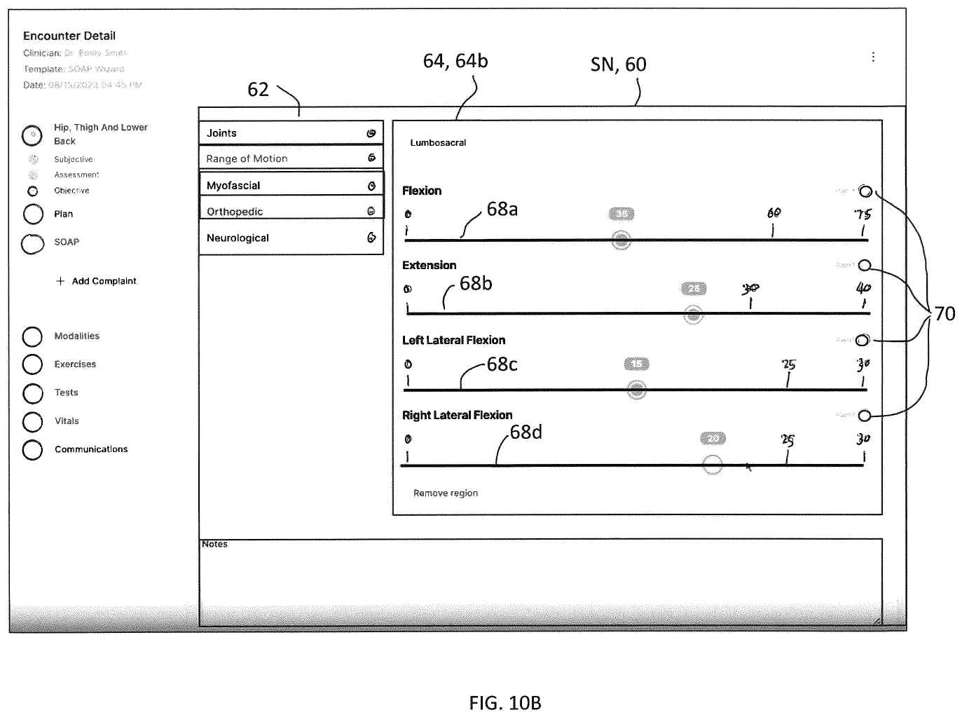

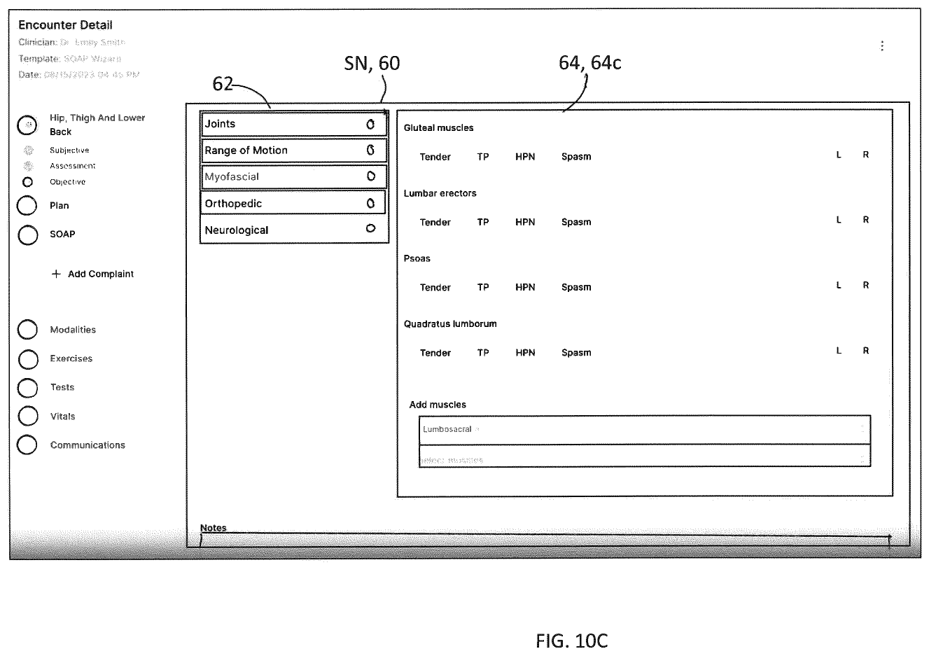

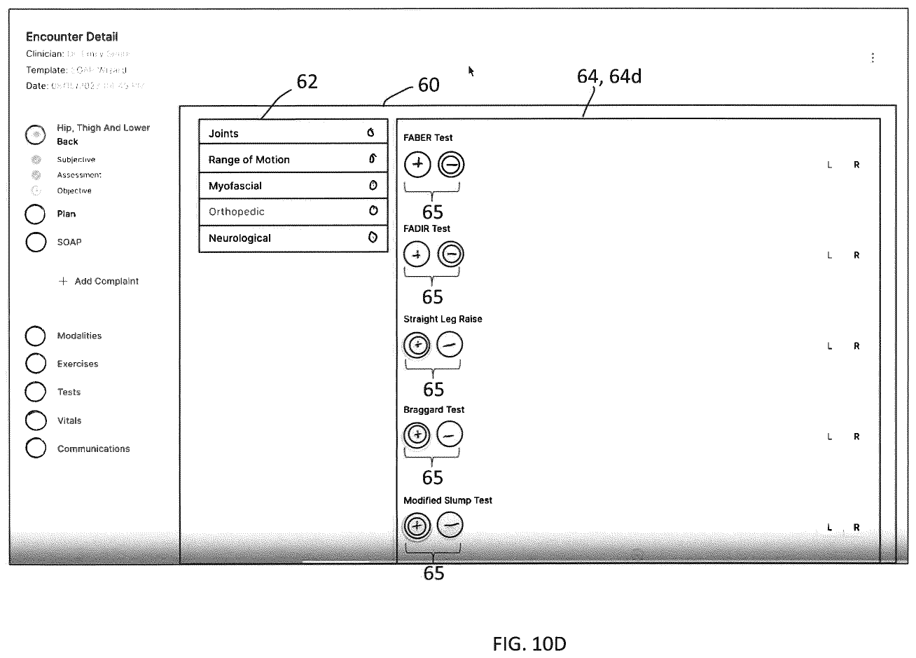

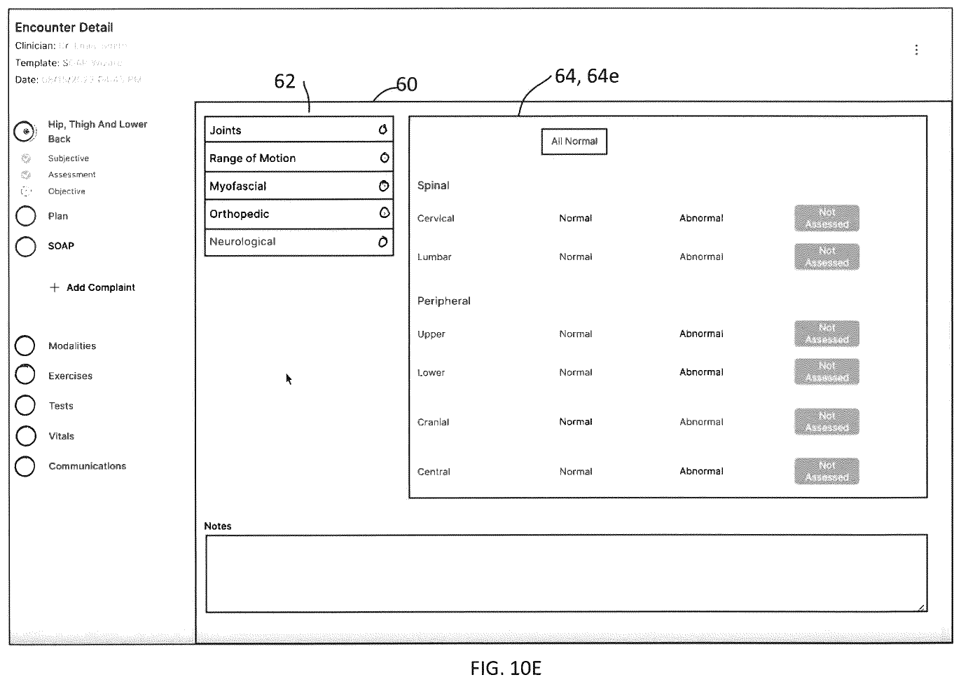

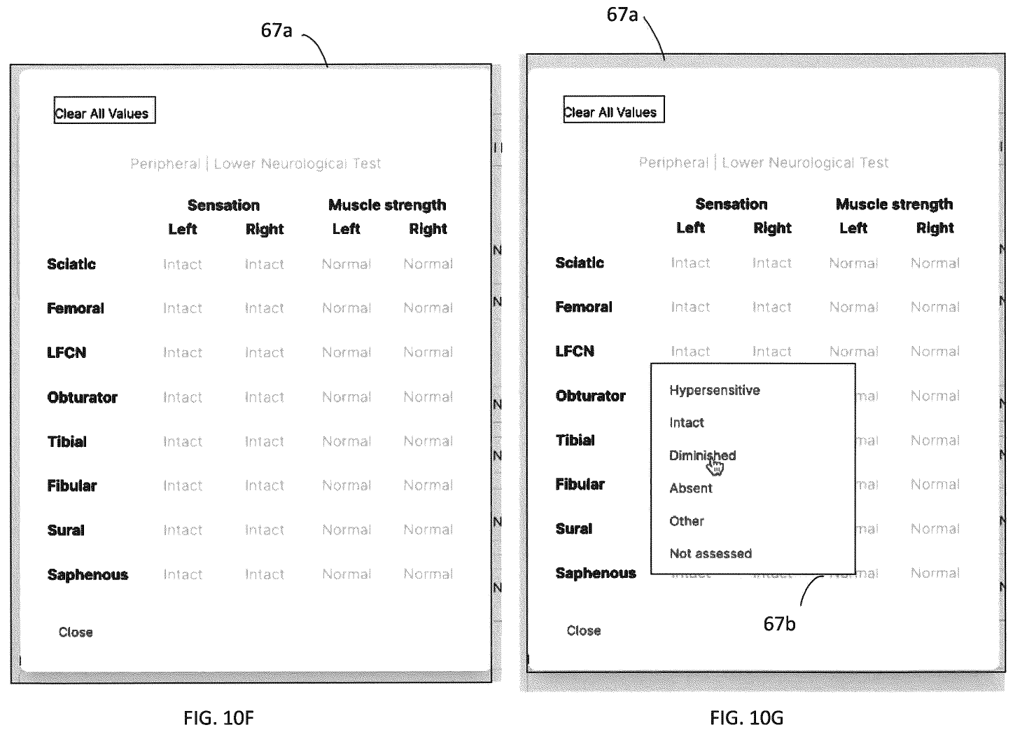

After the practitioner confirms that the assessment is completed to his/her satisfaction, the practitioner can move to the objective portion of the SOAP note by clicking on “objective” in the SOAP note menu 48. FIG. 10 shows the objective section 60 of the patient encounter record. In this section, the practitioner will document observations and different clinical tests performed with the patient. In this section, the practitioner can document the results of any tests performed and observations made. Initially, the system will propose tests to perform and observations to make based on the information from the assessment portion of the SOAP note. The observation potion includes an observation menu 62 and an observation window 64. A note box 66 is below the observation window 64. The observation menu is pre-populated with objective examinations prioritized by the condition reference, which can be provided in descending order of predicted diagnostic yield. In the chiropractic field. The observation menu 62 enables the practitioner to select among the various categories of tests that can be administered. These test categories include “joints”, “range of motion”, “myofascial” “orthopedic”, and “neurological”. As can be appreciated, these test categories are designed for use by a chiropractic practice. A different practice may have a different set of test categories. The note box provides an area into which the practitioner can enter notes to supplement the information for each of the tests. This notes section can be tagged such that it will automatically carry over to the objective information section of a SOAP note for a next visit for this complaint.

As seen in FIG. 10A when “joints” is selected, the system displays, in the observation window 64, a “joint information” panel 64a containing tabs relating to the different joints in the body. As seen, the “joint information” box 64a includes tabs for “spine, “head”, “ribs”, “shoulder” “elbow”, “hand and wrist”, “hip and pelvis”, etc. In this example, the patient complained of hip, thigh, and back issues, so upon selection of “joints”, the system displayed an image of a spine. The practitioner can then select on the diagram the affected area of the skeleton. In this example, the practitioner can select specific sections of the spine. If desired, the practitioner can select other joints to provide information regarding those joints, by clicking on the boxes for the other joints. For example, the practitioner may want to review the tab relating to the “hip and pelvis”

As seen in FIG. 10B, when “range of motion” is selected, the system displays, in the observation window 64, a range of motion panel 64b providing the practitioner with slide bars 68a-d which allow the practitioner to input results from range of motion tests, including flexion, tension, left lateral flexion, and right lateral flexion. To the right of each test, the screen includes a radio button 70 which allows the practitioner to indicate if the patient experienced pain with any of the tests. If the patient did experience pain, the system could provide a pop-up window which allows the practitioner to quantify the level of pain, for example, on a scale of 1 to 10.

As seen in FIG. 10C, when “myofascial” is selected, the system displays in the observation window 64 a myofascial text panel 64c displaying specific muscles the system suggests be tested. The muscles displayed are selected by the system based on the diagnosis and will be the muscles typically tied to the particular diagnoses. The muscles to test is thus populated from the “clinical evaluations” data of the condition reference. If desired, the myofascial screen provides the practitioner with the option to remove muscle(s) that will not be tested or to add additional muscles to the practitioner wants to test. Muscles can also be added from a selection list or can be typed in. The practitioner can indicate symptoms for the particular muscles and which side (left or right) of the body is affected. In the context of a chiropractic practice, such symptoms could be “tender”, “TP” (trigger points present), “HPN” (hypertonicity present), and “spasm”.

As seen in FIG. 10D, when “orthopedic” is selected, the system displays an orthopedic panel 64d in the observation window 64. In the orthopedic panel 64d, the practitioner will document orthopedic findings from relevant tests. The tests, shown in the information screen will be auto-populated by the system based on in information in the condition reference. The practitioner will have the option to remove tests or to add additional tests. Each test is provided indicia 65, such as a “⊕” and “\textcircled{−}”, to indicate if the test results are positive or negative. Further, the system indicates, based on the diagnosis, which tests are typically negative and which are typically positive, for example by providing a second circle around the “⊕” or “\hat{−}” or providing the “⊕” and “\textcircled{−}” in colors that indicate what is normally positive or negative, as appropriate. However, the practitioner will then enter the actual results by selecting the appropriate “+” or “−” indicia, to record the test results.

FIG. 10E shows the neurological panel 64e which is displayed when “neurological” is selected from the objective menu 62. Neurological test findings will be documented in this panel. As with the other panels, the neurological tests set forth in the neurological panel are pre-populated by the system for the specific diagnosis using the condition reference. As the tests are conducted, the practitioner can note if the results are “normal” or “abnormal”. If any test is not run, the practitioner can select the “Not Administered” button at the right side of the list of tests. If any of the tests comes back abnormal, the system will show the table 67a in a pop-up window, as shown in FIG. 10F. The table 67a initially displays default normal values which can be edited using a drop down list 67b as shown in FIG. 10G to enter abnormal value resulted from the test. In the example shown, “intact” is the normal response or value. the abnormal values include “hypersensitive”, “diminished”, “other”. If a particular test is not run, the practitioner can note that the test was “not assessed”.

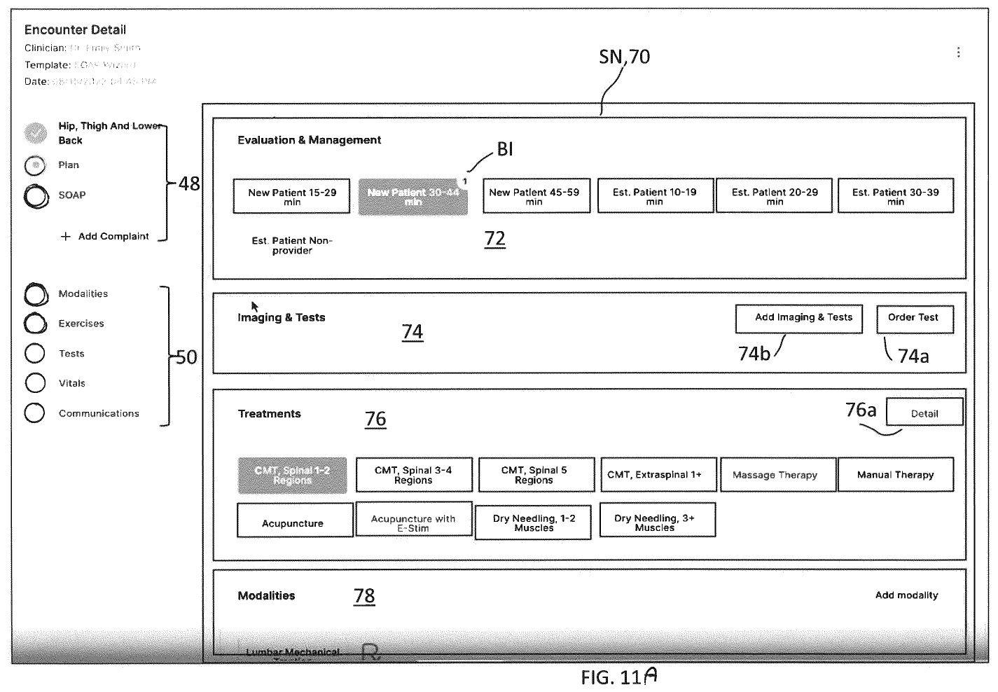

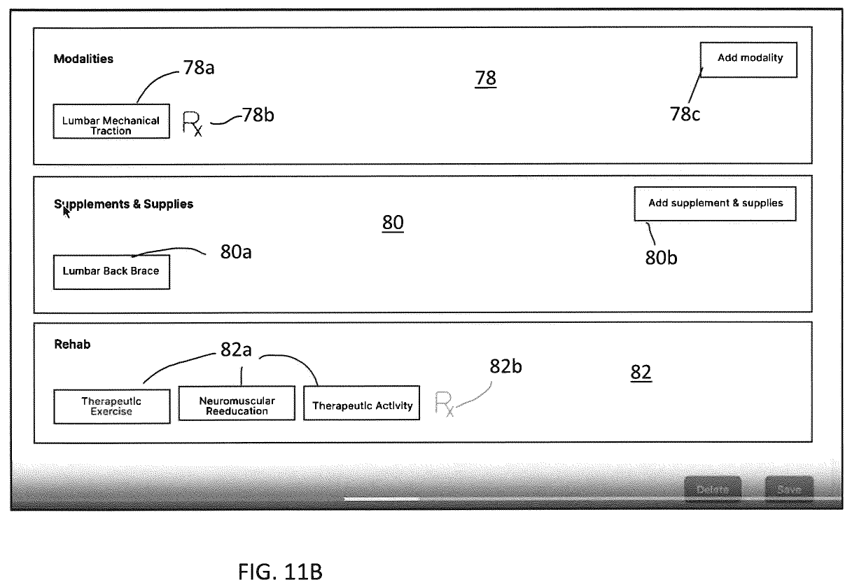

Once all the tests have been run, and the objective information has been entered, the practitioner can click on “plan” in the SOAP menu 48. The system will then display a plan window 70 (FIGS. 11A,B) in the SOAP note window SN. The plan window 70 includes an evaluation and management section 72, an imaging and tests section 74, a treatments section 76, a modalities section 78, a supplement and supplies section 80, and a rehab section 82. The information initially populated in the various sections of the plan by the system is based on the condition (i.e., diagnosis) and the assessment and objective information collected in the assessment and objective portions 56 and 60 using the condition reference 20.

In the evaluation and management section 72, the practitioner can select the type of evaluation conducted. In the illustrative example, a “New Patient, 30-44 min” evaluation is shown selected. In addition, the selected evaluation has a billing indictor BI in the form of a “1” in a circle (i.e., “\textcircled{1}” at the top right of the selected evaluation). This notes that the evaluation is marked for one billing unit. The system includes, can sync with, or communicate with, a financial module which will receive the billing information. The billing module can then prepare an appropriate claim to send to the patient's insurance provider or include in a bill to the patient.

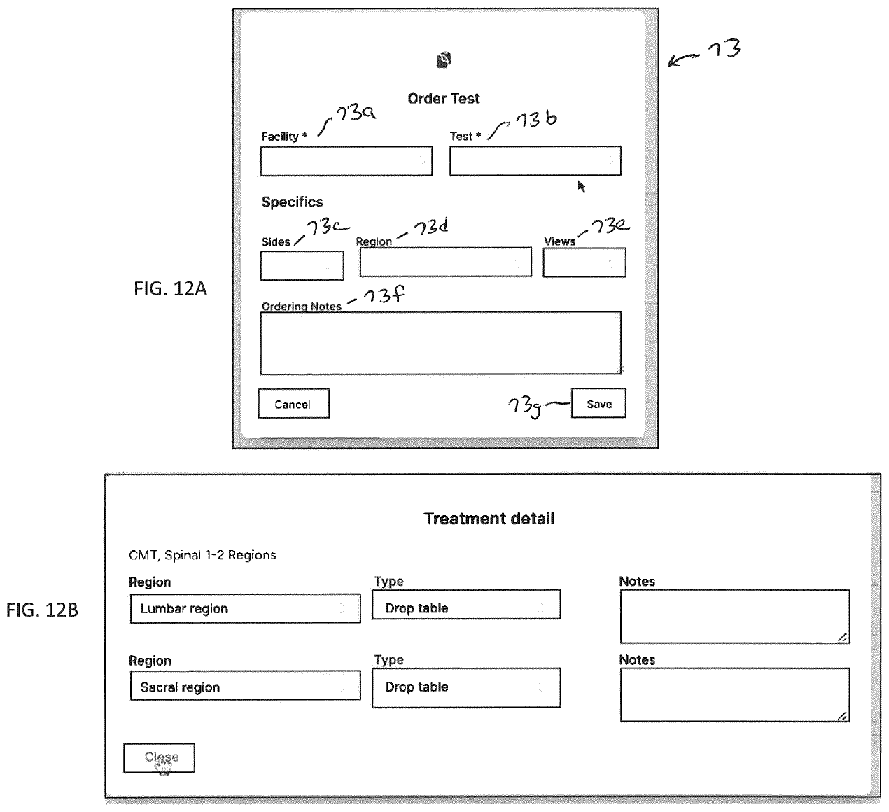

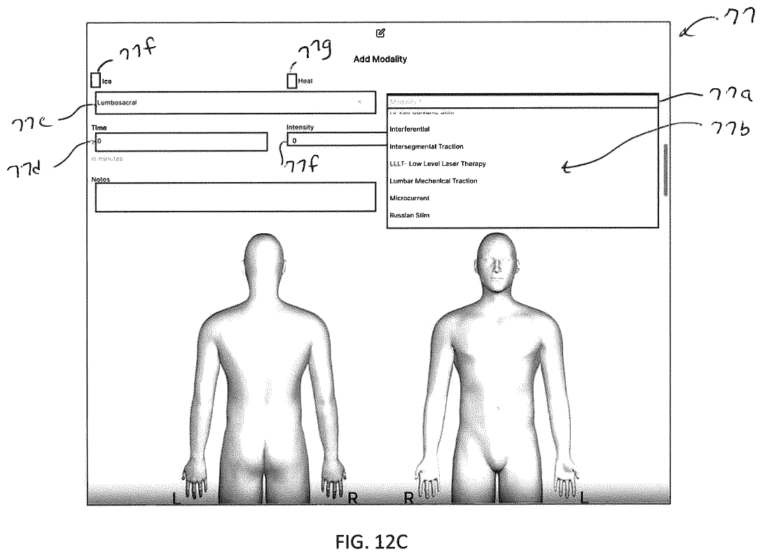

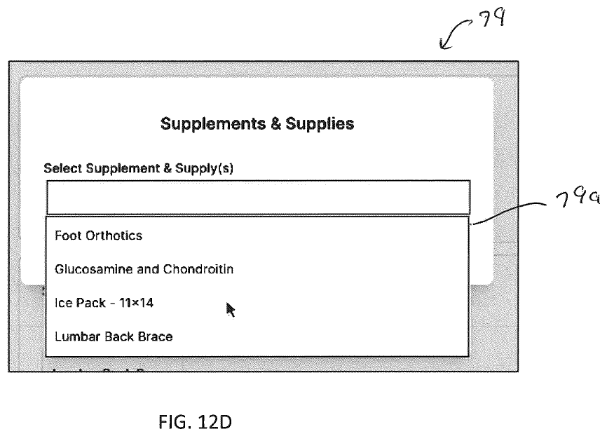

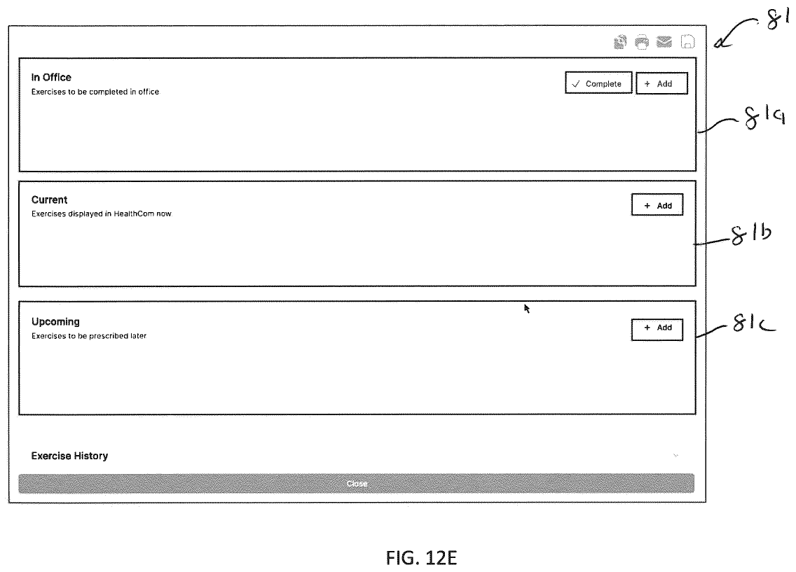

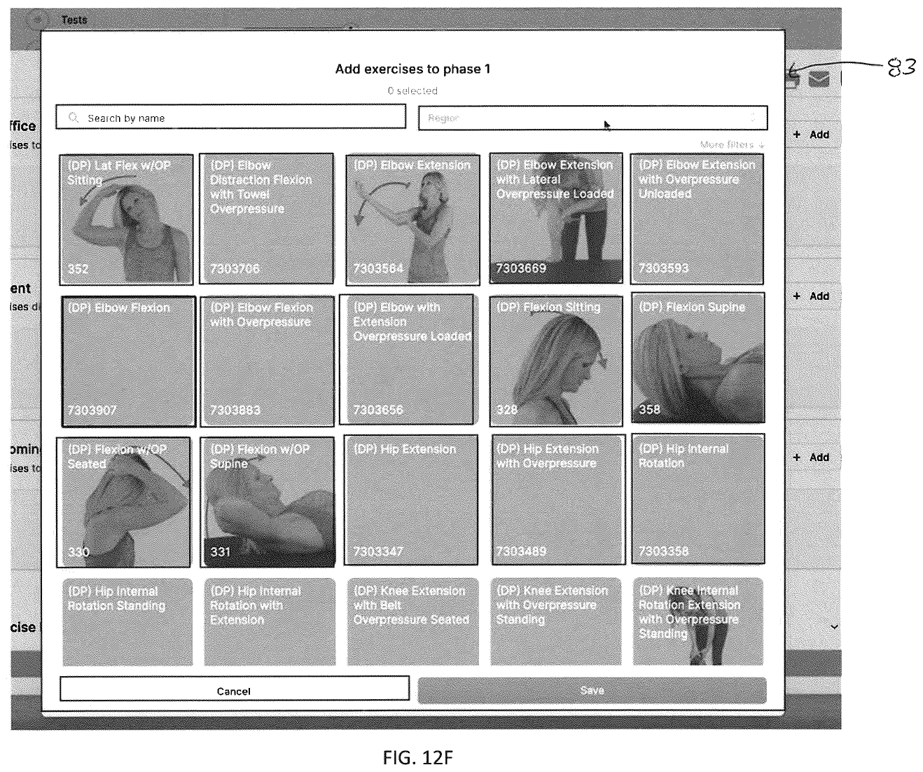

In the test portion 74 of the plan 70, the system can propose selected tests or images (e.g., x-rays) to run. The practitioner can confirm the tests suggested by the system by clicking the “Order Test” button 74a. Additionally, the practitioner can click the “Add Imaging & Tests” button 74b,to upload to the record additional tests and imaging that the practitioner might want. Clicking the “Order Test” button 74a will cause the system to display the order test pop-up window 73 shown in FIG. 12A. The Order Test pop-up window includes a facility selector 73a to select the facility where the imaging tests should be run. This can be in the form of a drop down list. The window 73 also provides the practitioner with selectors 73b-e which, respectively, enable the practitioner to select the test type (x-ray, CT Scan, MRI, thermal scan, etc.), the sides of the body (left, right, or both) to be imaged, the region of the body to be imaged (cervical, shoulder, elbow, etc.), and the number of views to be taken. A note box 73f allows for entry of any note the practitioner may want to enter related to the imaging to be performed. For example, the practitioner could note that a specific feature is being monitored. Clicking the “Save” button 73g will confirm the test order, and will save the information to plan of the patient's EMR. It will also save the test information in the “Test” module (selectable in the module menu 50), as will be discussed below. Notably, only the tests and imaging that are ordered become part of the SOAP note and patient's EMR. Thus, if a specific test or imaging protocol is suggested by the system, but not ordered by the practitioner, it will not become part of the record. As various imaging and tests are ordered, they are entered in the plan section and are added to the tests module (selectable in the module menu 50).

The treatments section 76 of the plan (FIG. 11A) is auto-populated by the system based on the diagnosis recorded in the assessment portion and the test results recorded in the objective portion, using the treatment techniques data in the condition reference. The treatments are proposed based on accepted treatment guidelines. FIG. 11A illustratively shows “CMT, spinal 1-2 regions” shown as being highlighted, thereby indicating that manipulations were made to the lower portion of the spine. The treatments that are not highlighted (such as CMT, Spinal 3-4 Regions”) were not (or have not yet) been made. As with the Evaluation & Management section, the individual treatments can be tagged for billing purposes. Clicking the “Detail” button 76a in the “treatments” section brings up a Treatment Detail pop-up window 75 shown in FIG. 12B. This pop-up window enables the practitioner to indicate the regions manipulated and the type of manipulation performed, and to add any notes in the “notes” box that the practitioner feels are warranted.

The modalities section 78 of the plan 70 (FIG. 11B) is initially auto-populated with modalities 78a suggested by the system based on the diagnosis recorded in the assessment portion and test results recorded in the objective portion. As is known, in the chiropractic field, “modalities” refers to therapies such as ultrasound, traction, etc. The modalities are suggested by the system based the modalities information in the condition reference 20 which, as has been noted, relies on the standard of care. The modalities in FIG. 11B are not highlighted. A suggested modality can be selected by clicking on the initially displayed modality. Selecting a displayed modality will cause the modality to be prescribed. Additionally, the practitioner can select any other modality by clicking on the Rx button 78b or the “add modality” button 78c in the modality section. Clicking on either the “Rx” or the “add modality” button will cause the system to display the “add modality” pop-up window 77 shown in FIG. 12C. The “add modality” pop-up window 77 includes a modality-type box 77a to indicate the type of modality. The Modality-type box, when entered, activates a drop down list 77b of modalities which can be selected. The modalities which can be selected are part of the ‘Clinical Skills” in the Main Menu M. The Add Modality pop-up window further includes a location box 77c to indicate where the modality is to be performed, boxes 77d, e to indicate the duration and intensity of the modality, and boxes 77f,g to indicate if ice or heat should be applied during the modality. The pop-up also includes images of the front and back of a body to enable the practitioner to provide additional indications regarding the modality (such as were stimulation is to be provided when traction is prescribed). As the modalities are entered in the plan section, they are added to the modalities module (selectable in the module menu 50). When the modalities are completed, the person performing the modality will note such in the modalities module, and that will then be updated in the note. Thus, the note is updated without the need for someone to later enter the note.

The Supplement and supplies section 80 of the plan 70 is auto-populated by the system based on the diagnosis and tests with supplies typically prescribed for the diagnosed condition. The proposed supplements and supplies are shown in boxes 80a which the practitioner can select by clicking on the indicated supply box 80a. By not clicking on the displayed supply will result in the supply not being prescribed. Additional supplements and supplies can be added by clicking on the “add supplement & supplies” button 80b. This will activate a “supplement & supplies” pop-up window 79 shown in FIG. 12D which allows the practitioner to select other supplies or supplies that the practitioner may want to prescribe. The various supplement & supplies can be selected from a drop down list 79a. The list of supplement & supplies is fueled by a supplement & supplies data structure.

The last section of the plan 70 is the rehab section 82. The rehab section is used to prescribe exercises for the patient. The rehab section 82 is auto-populated by the system based on the information from the subjective, assessment, and objective sections of the SOAP note with exercises typically prescribed for the diagnosed condition. The proposed exercises are shown in boxes 82a, and the exercises can be prescribed by clicking on the exercises. Additional exercises can be prescribed by clicking on the Rx symbol 82b. Clicking on the Rx symbol activates the exercise pop-up window 81 shown in FIG. 12E, which allows the practitioner to prescribe additional exercises to be performed by the patient. The system can auto-populate the exercise pop-up with exercises typically prescribed for the diagnosed condition. These exercises would be chosen by the system from exercise in the exercise data structure. Additionally, exercises can be selected using the exercise selector pop-up window 83 shown in FIG. 12F. As seen, the exercise selector pop-up window displays a plurality of exercises in an array, with each exercise being denoted by name and with an illustrative picture. The various exercises can be selected from the menu and added to the various boxes in the “exercise” prescription pop-up of FIG. 12E. The practitioner can add the exercises to an “in office” section 81a, current section 81b, and/or an “upcoming” section 81c. Exercises denoted as being “in office” are intended to be performed by the patient prior to leaving that day; exercise denoted as “current” are to be performed by the patient at home according to a prescribed schedule; and exercises denoted as “upcoming” are to be performed at a later date. When an exercise is prescribed, it will be highlighted in the SOAP Rehab section 82. Additionally, it will be added to the exercise module (selectable in the module menu 50).

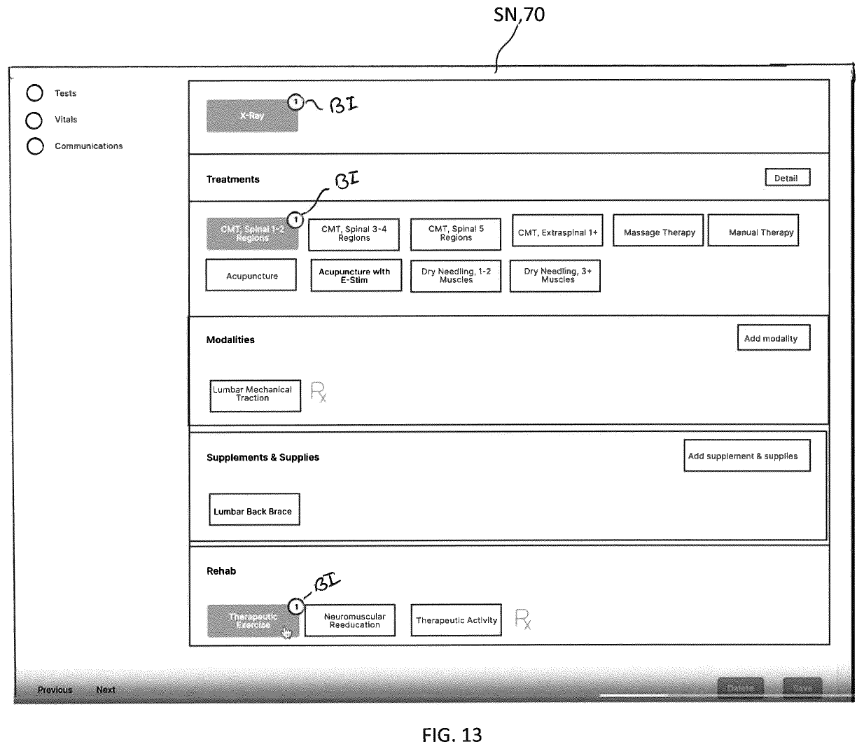

Upon completion of the rehab section 80, the plan section 70 of the patient EMR can appear as shown in FIG. 13. Here, the practitioner has clicked on selected entries that indicate actions that were prescribed during the patient visit. In the illustrative view of FIG. 13, this includes the X-ray, the CMT, and an exercise. There are all indicated by a circled “1” over the highlighted square. These “1”s are billing indicators BI. As noted above, the system includes, can sync with, or communicate with, a financial module which will receive the billing information. The billing module can then prepare an appropriate claim to send to the patient's insurance provider or prepare a bill for the patient for the actions taken during the patient encounter.

Once the plan has been completed, the practitioner can review the various selections in the module menu 50. As noted above, the module menu 50 includes selections for “modalities”, “exercises”, “tests”, “vitals”, and “communications”. The “modalities”, “exercises”, and “tests” panels are populated by the selections made in the plan 70 of the note SN. The practitioner or delegated staff can navigate among the five panels of the module menu 50 by clicking on the selected title in the menu to carry out or perform the actions (modalities, exercises, or tests) that were designated to be performed in the office.

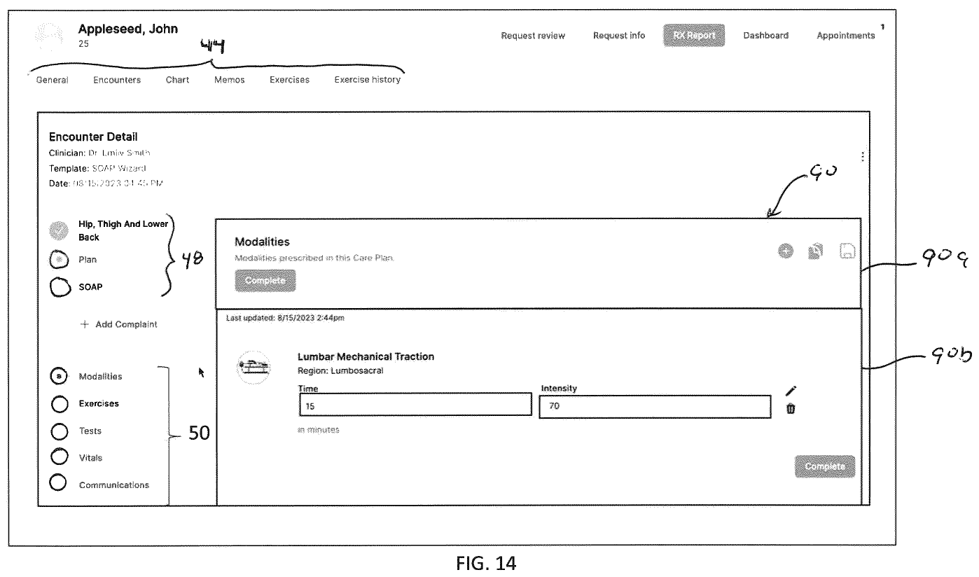

FIG. 14 shows the “modalities” panel 90, which includes a top portion 90a showing the modalities prescribed in the plan. The modality shown in this top portion is the modality prescribed in the plan portion 70 of the note. A modalities history section 90b is below the prescribed modalities section to show what modalities had been previously prescribed. The practitioner or delegated staff member can click on the “complete” button adjacent the various modalities shown to indicate that the specific modality has been completed.

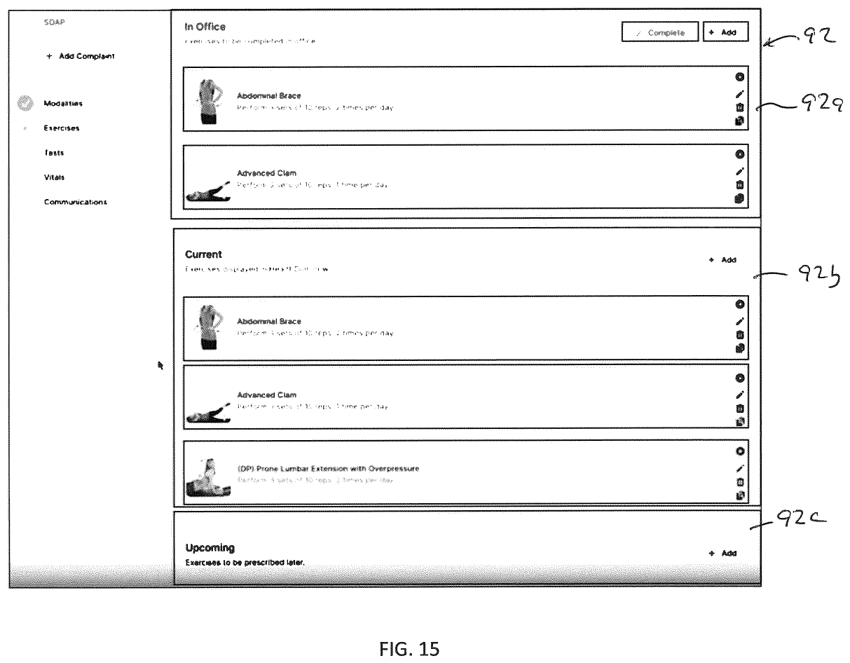

FIG. 15 shows the Exercises module 92. As seen, the exercises tab is populated with the exercises prescribed in the plan, which are split among the three sections “in office” 92a, “current” 92b, and “upcoming” 92c. The therapist can review the exercise tab and train the patient on the exercises or have the patient conduct the in-office exercises. When, for example, the in-office exercises are completed, the therapist can click the “complete” button to indicate that the in-office exercises have been completed. In addition, the therapist can, if appropriate, mark any exercises completed in office with a billing indicator BI, such that a proper claim can be made with the patient's insurance company so that the patient can be billed.

The modalities and exercise modules can be relevant for subsequent patient encounters. When the patient returns for a second visit, the therapist can navigate to the modalities and exercise modules to see what modalities and exercises were previously prescribed, and if any modalities or exercises need to be performed prior to meeting with the practitioner. After any such modalities or exercises have been completed, the therapist can mark the specific modality or exercise as complete (and add a billing indicator if appropriate). This information will then be included in the SOAP note for the subsequent visit.

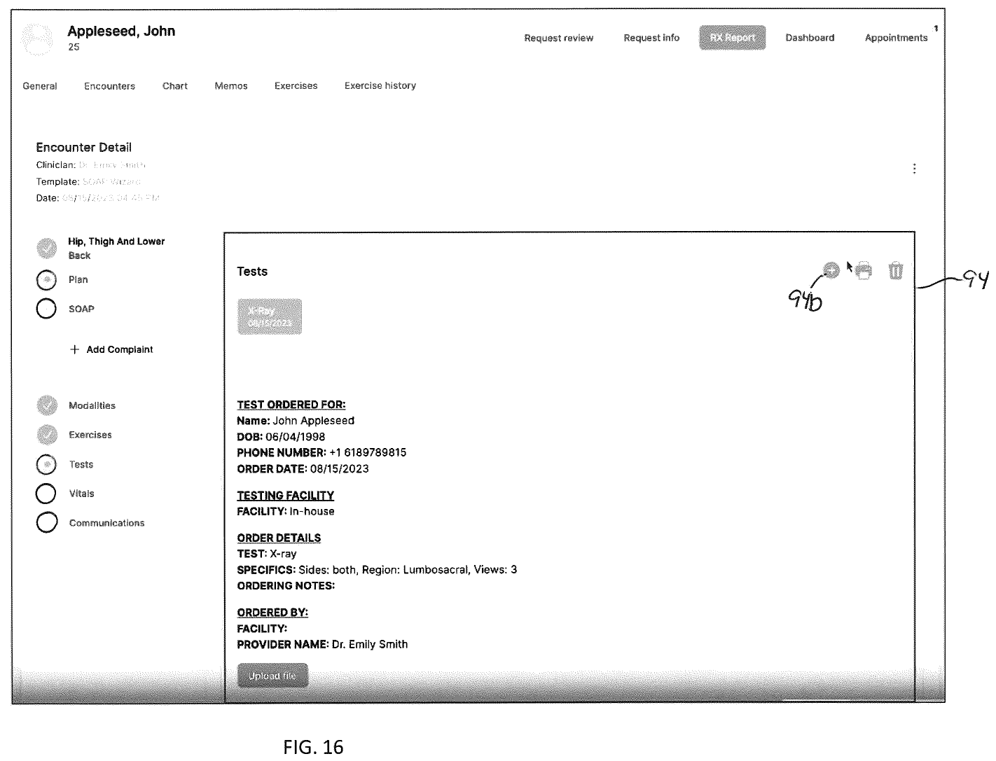

FIG. 16 shows the “test” module 94 of the EMR. The test module will show the tests ordered during the plan, along with the test details. Test results can be uploaded to the record using the “upload files” button 94a below the test details. Additionally, by clicking on the +button 94b, additional tests can be ordered.

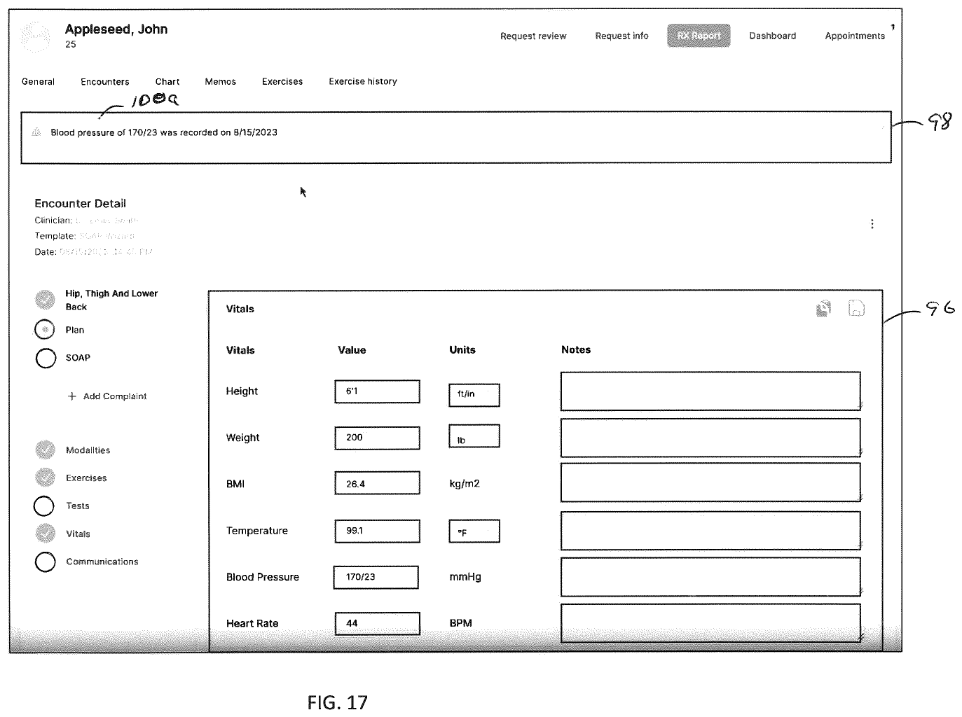

FIG. 17 shows the vitals module 96 of the EMR. In this module, the patient's vitals, i.e., height, weight, temperature, BMI, blood pressure, heart rate, etc. can be entered into the system. In the illustrative example shown, the patient has a blood pressure of above 160, which is high. The system thus generated a warning contained in a box 98 which appears at the top of the vitals screen. As seen, the warning box 98 includes the subject matter (i.e., high blood pressure), the value, and the date measured. This will enable the practitioner to monitor the particular vital. This warning will show at the top of all screens of the EMR so that the provider is aware of the existence of the out-of-range vital. The warning can be highlighted so that it stands out. For example, it can be surrounded by a yellow box. Additionally, the warning can include a warning icon 100a, shown in the form of an exclamation mark (“!”) inside a triangle (i.e., “ [symbol] ”).

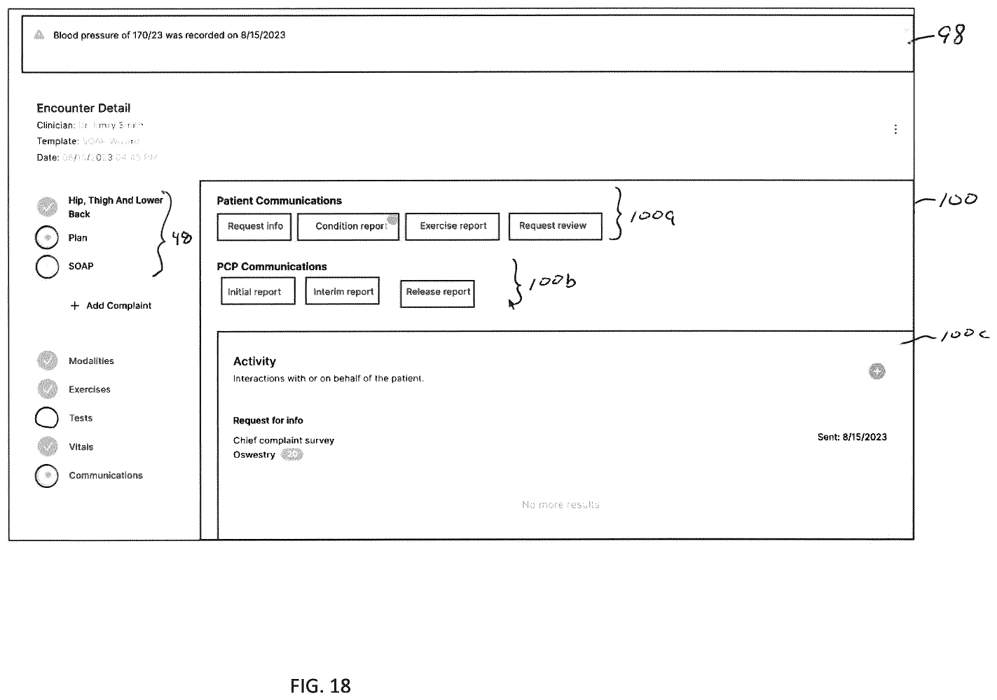

FIG. 18 shows the communications module 100. In a top portion 100a of the communications module, the practitioner can manage all communications with the patient, including information requests, condition reports, and exercise reports. In addition, a review can be requested. Below the patient communications section, the provider can, in a PCP communication section 100b, manage PCP communications with the patient. The PCP communications section 100b enables the practitioner to generate initial, interim, and release reports quickly. Below the PCP communications section, the communications tab includes an activity section 100c which summarizes all communications with the patient for the particular encounter.

The system can also be provided with a Scheduling and Goals module. This module (not shown) would enable the practitioner to set the number of appointments believed to be necessary for the condition and the frequency of the appointments. These appointments would automatically be added to the calendar. The system could then remind the practitioner when it is time to reevaluate the condition complained of to more precisely document the change (improvement) in the condition. The Goals portion of the module would indicate what degree of improvement is expected.

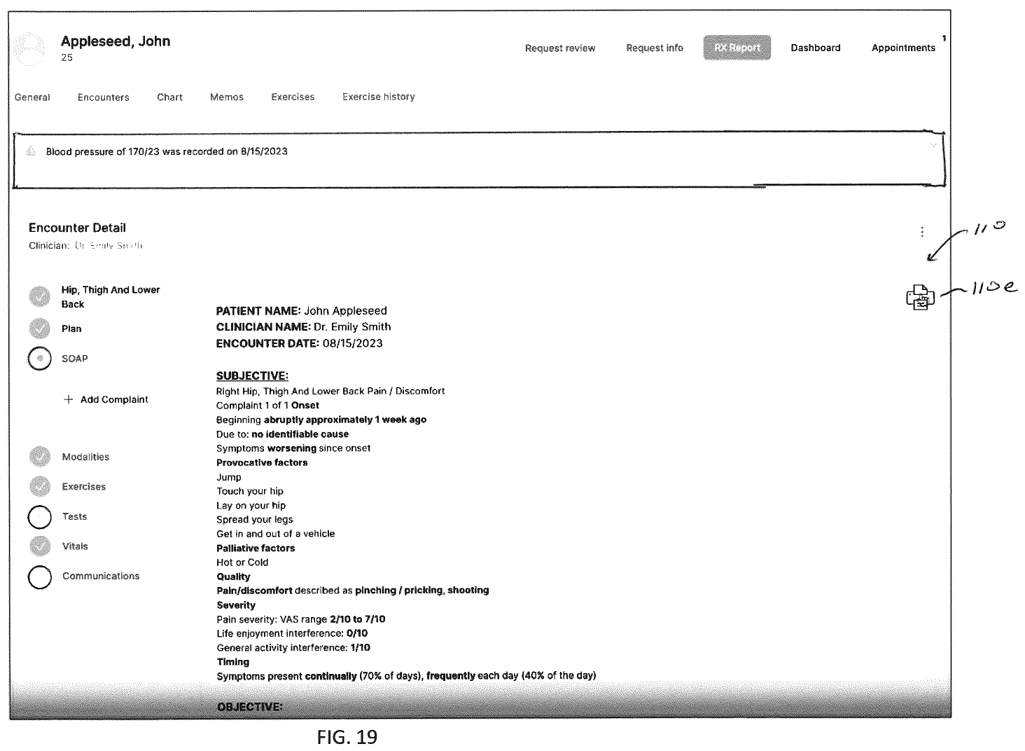

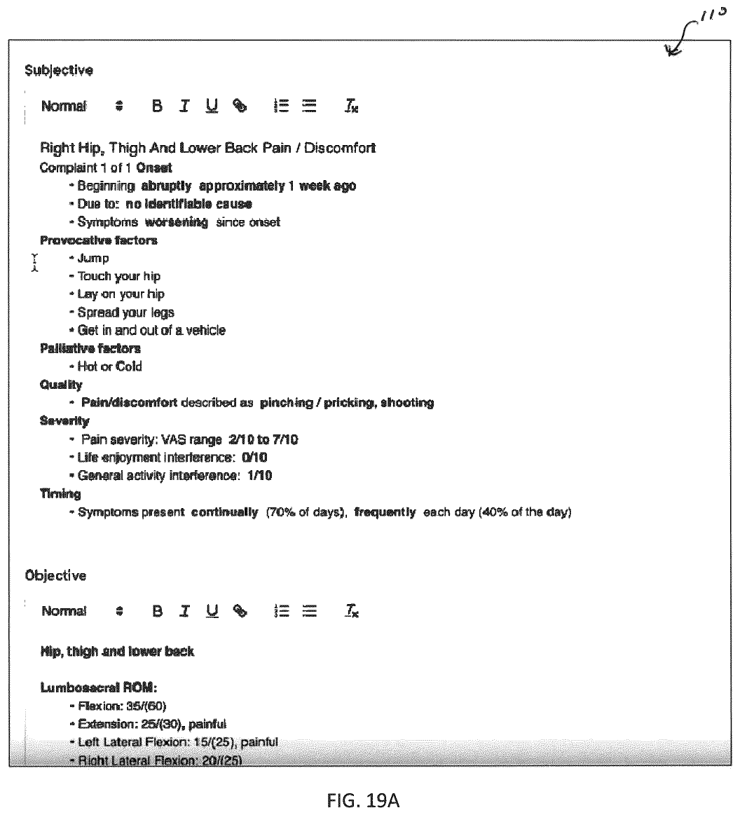

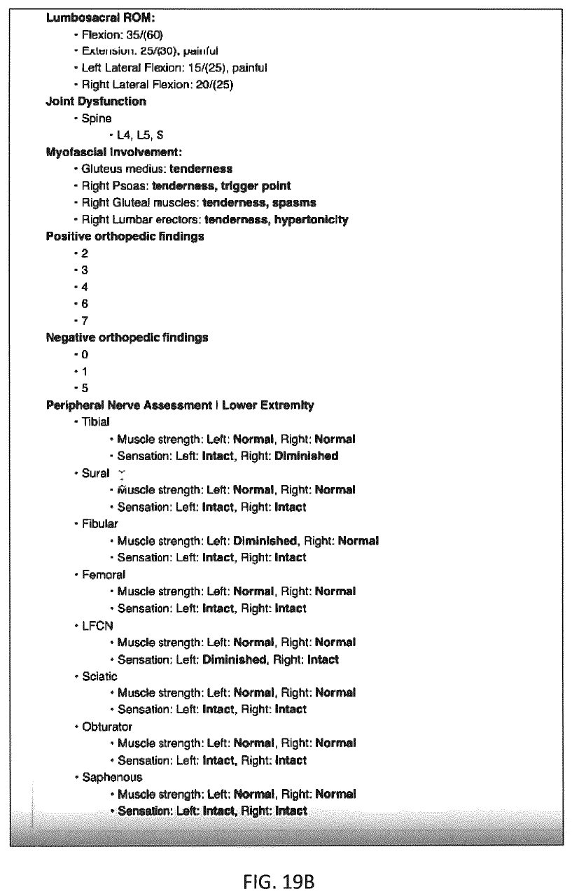

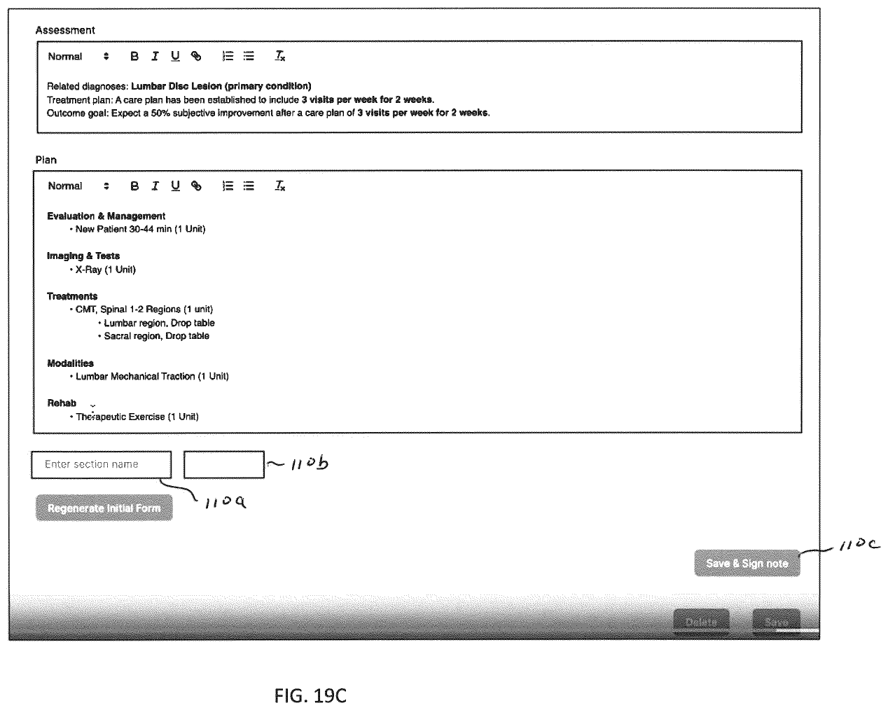

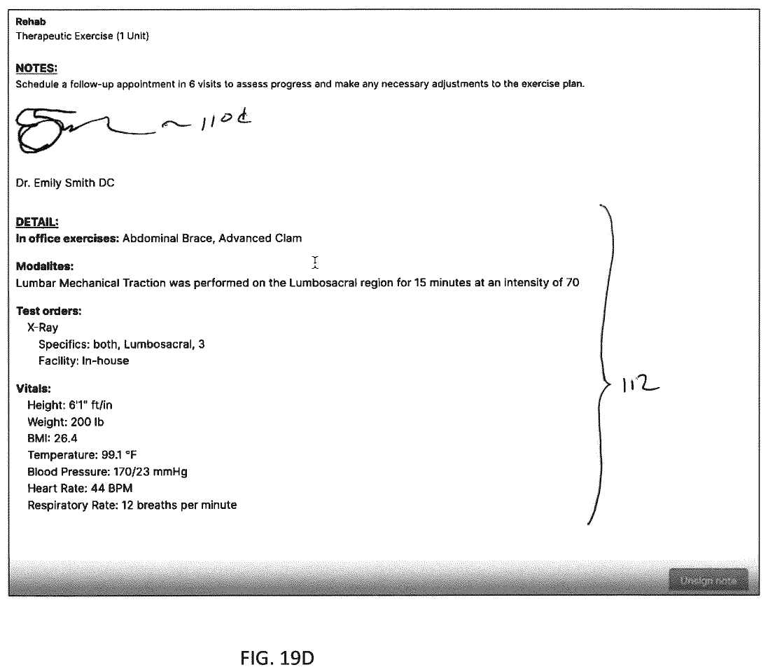

Once everything has been completed, the practitioner can navigate to the SOAP report by clicking on the SOAP selection in the SOAP menu 48. A completed SOAP note 110 is shown in FIGS. 19 and 19A-C. The SOAP note contains all the information that was entered into the system both by the patient from the questionnaire and by the practitioner during the patient encounter in the assessment, objective, and plan sections of the SOAP note, as described above. As the practitioner reviews the SOAP note, the practitioner will have the option to edit any section of the note. Additionally, the practitioner can add one or more sections to the note. To do so, in the box below the “Plan” section, the practitioner can enter the new section name in the “enter section name” box 110a and click on the “add section” button 110b. (FIG. 19C) Doing so will open a text box (not shown) in which the practitioner can enter text based notes. When the practitioner is ready, the practitioner can click the “save and sign note” button 110c to save and sign the SOAP note. This will add the practitioner's signature 110d to the note (as shown in FIG. 19D) and save the SOAP note to the EMR for the patient.

The bottom portion of a signed SOAP note is shown in FIG. 19D. Below the signature 110d is a detail section 112 containing information documented in the modalities, exercises, tests, vitals, and communications modules. The practitioner will be able to later review the completed and signed SOAP note by selected “Encounters” on the EMR menu 44. The practitioner will then be provided with a list of encounters with the patient, allowing the practitioner to call up the SOAP note for any of the visits with the patient.

The completed SOAP note can be printed or downloaded by clicking on a print icon 110e (FIG. 19) at the top of the SOAP note. Selecting the print icon will display a list of reasons for printing the SOAP note. These can include, for example, “in-office use”, “patient request”, “insurance carrier”, “attorney”, “outside provider”, or “other”. When printed, the SOAP note will print together, but the detail section (below the signature) will print on its own (i.e., on a new page, separate from the last page of the SOAP note).

When the patient next visits the practitioner, a new encounter record (and thus a new SOAP note) will be generated by the questionnaire generator. The information from the SOAP note from the current encounter will be relied upon by the system to guide the questions asked by the system in the patient questionnaire. Stated differently, the system is designed to ask relevant and intuitive questions based on the prior input received from both the provider and the patient. In particular, the questionnaire generator will base the questions on the diagnosis and prescribed treatment plan as set forth in the prior SOAP note. The ability of the system to ask more targeted questions of the patient ensures that the intake process for subsequent visits by the patient is personalized and efficient by addressing the specific needs and conditions of the patient as previously indicated. This enables the system to generate a more meaningful subjective information section of the SOAP note for the next visit. As alluded to above, for that next visit, the practitioner will then be able to review the patient's answers to the updated questionnaire, to make the subsequent appointment more productive. As noted in the discussion of FIG. 7, the EMR menu 44 allows for the practitioner to navigate through the various sections of the EMR.

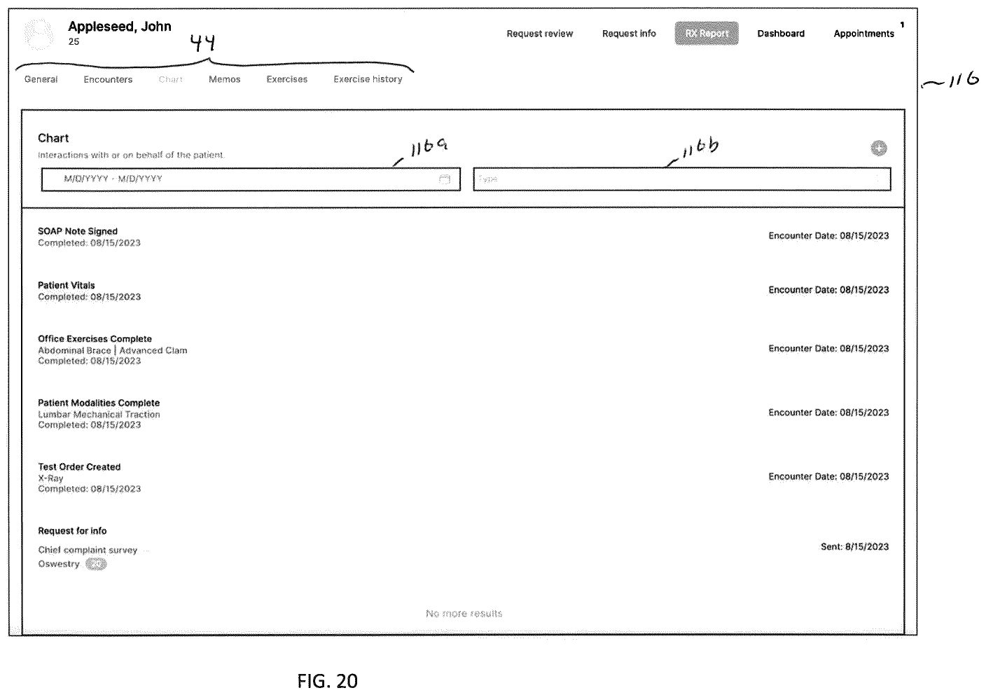

FIG. 20 shows a chart panel 116 of the patient's EMR which is accessible by selecting “chart” in the EMR menu 44 at the top of the EMR. The Chart panel displays, in list format, the interactions for the patient. The history can be searched, for example, by date or by type in the respective search boxes 116a,b. The “type” search will allow the provider to select, for example, SOAP notes, exercises, modalities, tests, memos, etc. The provider can then click desired items in the provided list to review selected items.

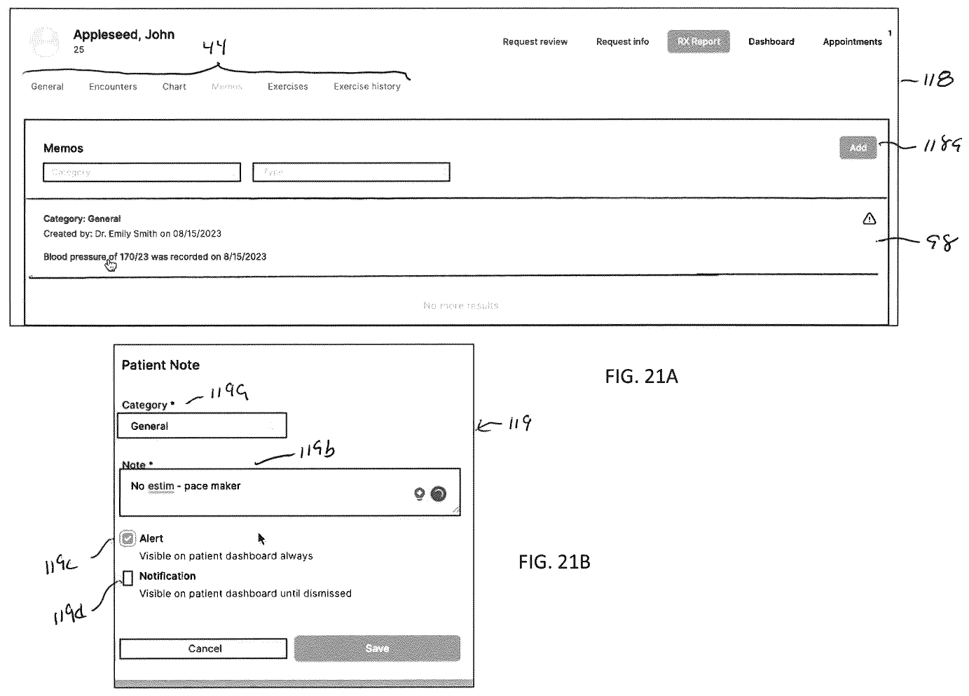

FIG. 21A shows a memo panel 118 of the EMR which is accessible by selecting “memo” in the EMR menu 44 at the top of the EMR. The memo panel shows the warning and notifications (such as the high blood pressure warning 98 from the vitals panel (FIG. 17)). Additionally, by clicking on the “add” button 118a, the practitioner can add a note to the file. This activates the add note pop-up 119 of FIG. 21B. The pop-up 119 includes fields 119a for the category (or type) of note and a free form text box 119b to enter note. Additionally, the pop-up can include an “alert” check box 119c that can be selected so that the note will display at the top of the various screens in the EMR. Such alerts are typically for long-term alerts. Alternatively, the practitioner can tag the note as being a “Notification” by checking the check box 119d. A notification will remain with the patient record and will appear on the dashboard for the record until the notification is dismissed (such as when an issue has resolved).

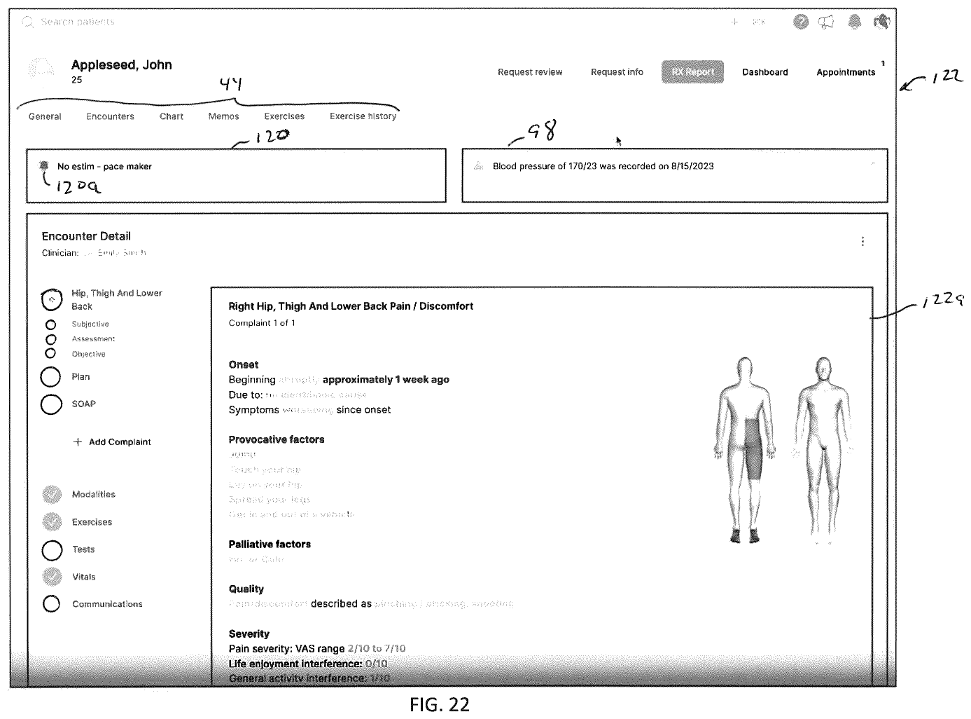

FIG. 22 shows the details of the “encounter” tab 122 of the EMR, accessed by selecting the “encounters” option in the EMR menu 44. As seen, the encounter tab includes an encounter detail panel 122a which summarizes the patient encounter and incudes all the information from the SOAP note. The tab also shows the note 120 that was entered into the memo and the warning 98 from the vitals panel. The note 120 includes an alarm icon 120a and can be surrounded by a red box to help denote it as an alert. The auto-generated warning 98 regarding blood pressure, on the other hand, includes a warning icon and can be surrounded, for example, by a yellow box.

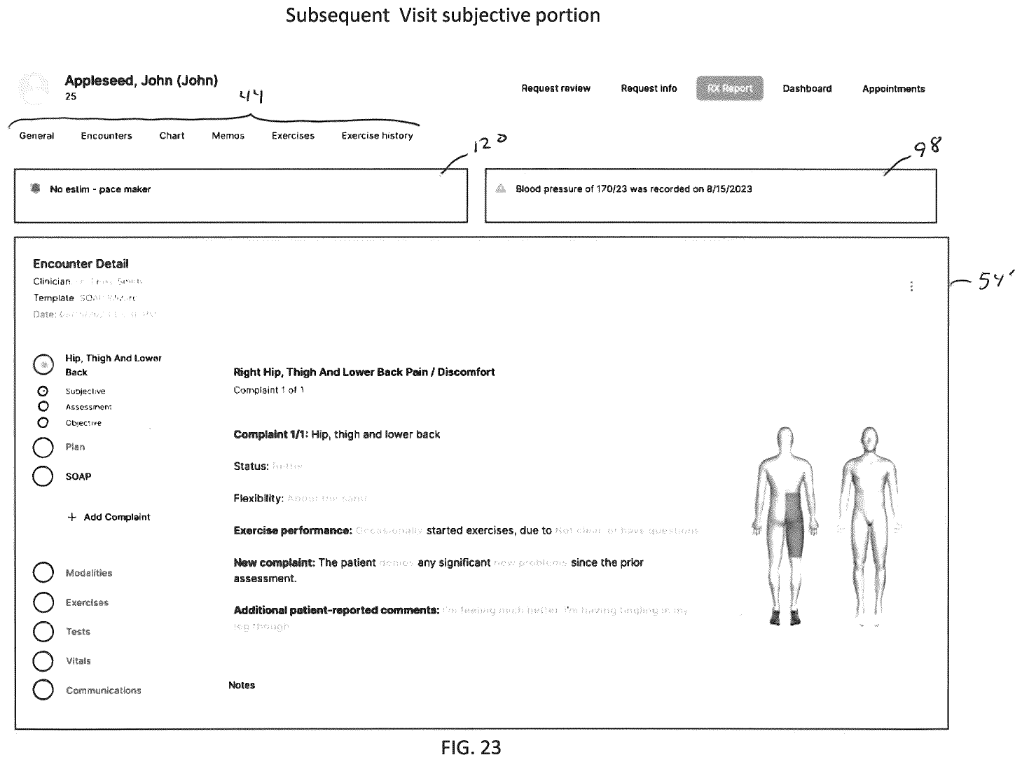

FIGS. 23-27 show the development of the patient's EMR and a second SOAP note for a second (or subsequent) visit for the same condition. In a second (or subsequent) visit, the patient will initially complete the patient questionnaire. As noted above, the questionnaire generator 22 will generate the questionnaire based on the information in the prior SOAP note. Thus, the patient will be presented with questions specific to his/her condition. For example, the patient can be presented with questions as to whether or not the symptoms are improving or worsening. The system will generate a new SOAP note for each visit. Thus, the patient's answers to the follow-up visit questionnaire will be used to populate the Subjective portion 54′ of a new SOAP note. FIG. 23 shows active screen AS of the EMR with the subjective portion 54′ of the note being selected. Initially, the system includes the warning 98 and alert 120 which were entered in patient's EMR in the prior encounter with the patient. Thus, the practitioner will immediately be alerted to the noted conditions which may be relevant to any testing and the continued treatment plan for the patient.

With reference to the subjective portion 54a of the new SOAP note, it can be seen that the questions presented to the patient in the subsequent visit questionnaire were different from the initial visit questionnaire (FIG. 8). The answers to the new questions for this subsequent visit are highlighted for the practitioner's convenience. If the patient indicated in the answers to the subsequent visit questionnaire that there was an additional complaint, the questionnaire generator would issue questions to the patient related to that additional complaint.

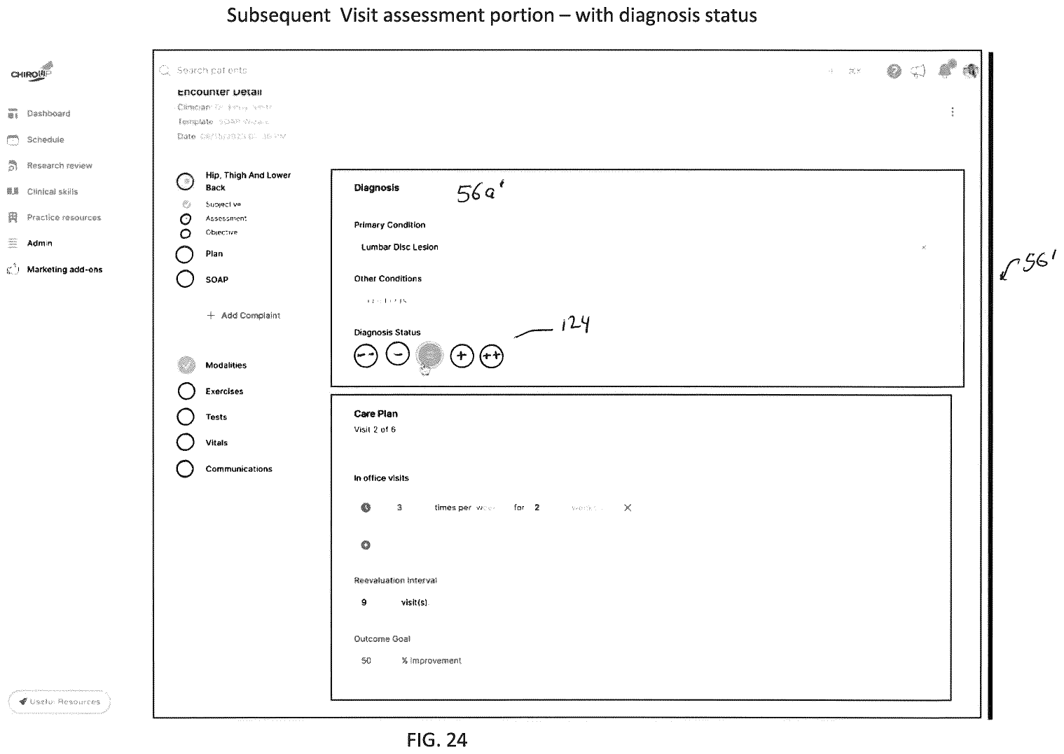

FIG. 24 shows the assessment portion 56′. The assessment portion displays the diagnosis, which is carried over from the prior SOAP note for this condition, in the diagnosis section 56a′ of the assessment portion. The diagnosis section 56a′ includes a diagnosis status section 124 in which the practitioner can note the status of the condition relative to the status of the condition from the prior visit. Here, the change in status is represented by a series of icons, illustratively “−−”, “−”, “=”, “+”, and “++”, which indicated, respectively, “much worse”, “worse”, “about the same”, “improved” and “much improved”. The selected icon is highlighted. As the practitioner reviews the information carried over from the SOAP note for the prior visit, the practitioner can modify, if necessary, the frequency of in-office visits, the reevaluation interval, and the outcome goal.

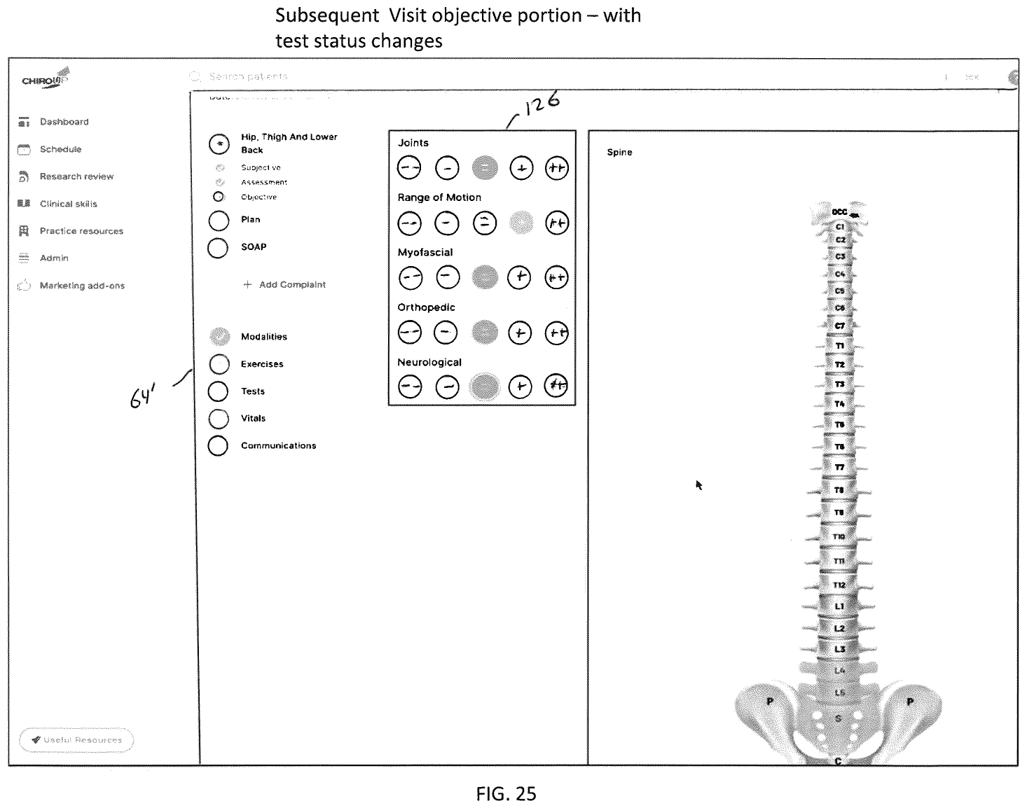

FIG. 25 shows the objective portion 64′ of the subsequent visit SOAP note. Again, the information (i.e., tests and observations) from the prior visit are carried over to this new SOAP note. The objective portion 64′ includes a test status box 126 which enables the practitioner to note the change in test results. As seen, the objective portion includes the image of the portion of the body tested (the spine in this instance) with the relevant portions of the skeletal structure highlighted to illustratively show what was tested previously. As with the diagnosis status, the change in test results are shown by a series of icons, illustratively “−−”, “−”, “=”, “+”, and “++”, which can be selected to indicate the degree of positive (or negative) improvement in the various tests conducted. In this section, the practitioner can conduct, and thus document, additional tests if deemed necessary. If a test conducted during the prior visit is deemed to be no longer relevant, the practitioner can note that the particular test was not performed. The reasons for not performing a test or for adding additional tests can be documented in a note field.

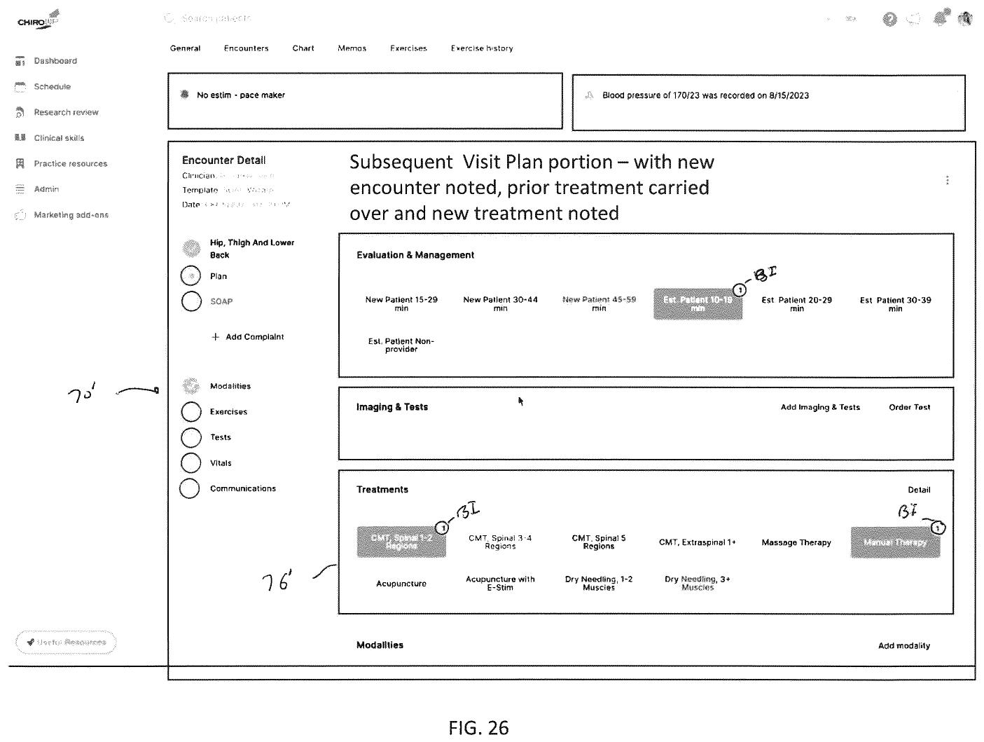

FIG. 26 shows the plan portion 70′ of the subsequent visit SOAP note. Here, the practitioner can indicate, for billing purposes, the type of visit. As seen, a billing indicator BI is added to the “Est. Patient 10-18 min” visit box. The system will carry over, from the prior visit, treatments that were prescribed and/or carried out at the prior visit. This is shown in the treatments section 76′. Here, the practitioner can see what prior treatments were prescribed and can, if appropriate, prescribe additional treatments. Additionally, the practitioner could opt not to carry out a treatment from a prior visit, by deselecting the treatment from prior visit. Billing indicators BI can then be added to treatments that are carried out in the office.

Each section of the SOAP note can be provided with a note box, as described above, which will allow the practitioner to enter notes relative to the particular section of the SOAP note. Once the visit with the patient is completed, the practitioner can review the subsequent visit SOAP note and sign it in the same manner as discussed above.

If there are further visits for the same complaint, individual SOAP notes would be generated for each of those visits in the same manner as just described for the second visit.

As can be appreciated, we have provided a SOAP note system which is fairly intuitive. Further, by relying on AI (through the condition reference) to suggest modalities, exercises, tests, and plans based on accepted treatment plans for the diagnosed condition the practitioner will be less likely to inadvertently miss a modality, test, or exercise that may be important to the patient's recovery from the complained of ailment.

As various changes could be made in the above constructions without departing from the scope of the invention, it is intended that all matter contained in the above description or shown in the accompanying drawings shall be interpreted as illustrative and not in a limiting sense.

1. An electronic medical record system comprising a patient portal, a practitioner portal, a server, and a non-volatile memory in which is stored an electronic medical record (EMR) data structure and a condition reference data structure, said condition reference data structure comprising data regarding diagnoses, care plans, treatment modalities, exercises, and tests; said patient portal and practitioner portal both being in communication with said server and being adapted to send information to, and receiving information from, said server;
said system including instructions stored in said non-volatile memory for generating a SOAP note for a patient encounter related to a patient complaint, said SOAP note comprising a subjective portion, assessment portion, objective portion, and plan portion; said instructions includes instructions for:
a) determining if the patient encounter is a first patient encounter or a subsequent patient encounter for the patient complaint;
b) generating a patient questionnaire and displaying said questionnaire on said patient portal; whereby:
b1) for an initial visit for a patient complaint, said system includes instructions for providing the patient with an image of a body and enabling the patient to indicate which portion of the body is involved in the patient complaint, and generating further questions to the patient in response to answers received from the patient; and
b2) for a second or subsequent visit for the same complaint, said system includes instructions for generating questions for patient as a function of information from a prior SOAP note for the patient complaint, wherein said questions generated elicit information from the patient regarding the status of the patient complaint;
c) receiving responses to questions in said questionnaire entered by a patient;
d) populating the subjective portion of the SOAP note with the patient responses to the questionnaire;
e) upon request, displaying the subjective portion of the SOAP note on the practitioner portal;
f) receiving a diagnosis from the practitioner and entering the diagnosis in the assessment portion of the SOAP note;
g) auto-populating a care plan in the assessment portion based on the diagnosis and care plan data in the condition reference data structure;
h) auto-populating the objective portion of the SOAP note with proposed tests for the practitioner to run and displaying the proposed tests on the practitioner portal, the proposed tests being selected based on the diagnosis and care plan data in the condition reference data structure;
i) receiving from the practitioner portal test result information entered into the practitioner portal by the practitioner for tests run and storing test data in the objective portion of the SOAP note; said test data comprising tests run and test results for the tests run; said tests run including at least a portion of the tests proposed by the system;
j) auto-populating the plan portion of the SOAP note with a proposed plan of treatment for the patient and displaying the proposed plan of treatment on the practitioner portal; the proposed plan of treatment being based on the diagnosis, the test results, and care plan data in the condition reference data structure;
k) receiving from the practitioner portal information regarding a prescribed treatment plan and storing said prescribed treatment plan in the plan portion of the SOAP note; said prescribed treatment plan comprising at least in part aspects of the proposed treatment plan;
l) generating a completed SOAP note from the information stored in the subjective portion, assessment portion, objective portion, and plan portion of the SOAP note and displaying the completed SOAP note on the practitioner portal;
m) enabling the practitioner to electronically sign the completed SOAP note;
n) upon receipt of an electronic signature for the SOAP note, storing the SOAP note as a SOAP note record for the particular patient encounter in an electronic medical record for the patient.

2. The electronic medical record system of claim 1, said system being adapted to:
determine if a vital statistic (such as blood pressure, weight, BMI, pulse, etc.) is abnormal, generate a warning regarding said abnormal vital statistic, and display said warning on all screens of an electronic medical record for the patient; or
enable the provider to enter an alert regarding a condition of the patient and display said alert on all screens of the electronic medical record for the patient

3. The electronic medical record system of claim 1 wherein said server is remote or local and wherein said non-volatile memory is remote or local.

4. The electronic medical record system of claim 1 wherein said patient portal and practitioner portal each comprise one or more of kiosks, laptop computers, tablet computers, cell phones, or combinations thereof.

5. The electronic medical record system of claim 1 wherein said system is adapted to enable the practitioner to request additional tests; wherein said test data includes said additional tests and results of said additional tests.

6. The electronic medical record system of claim 1 wherein said proposed plan of treatment comprises one or more of proposed imaging and/or tests, proposed treatments, proposed modalities, proposed supplements and/or supplies, and proposed exercises.

7. The electronic medical record system of claim 6 wherein said system is adapted to enable the practitioner to prescribe additional imaging and/or tests, treatments, modalities, supplements and/or supplies, and/or exercises not proposed by the system; wherein the prescribed plan of action comprises at least a portion of the proposed imaging and/or tests, proposed treatments, proposed modalities, proposed supplements and/or supplies, and proposed exercises and the additional prescribed imaging and/or tests, treatments, modalities, supplements and/or supplies, and exercises.

8. The electronic medical record system of claim 1 wherein upon completion of the objective portion of the SOAP note, test data comprising tests run and test results is synced to, or stored in, a test panel; said test panel of the EMR containing a history of tests run on the patient.

9. The electronic medical record system of claim 1, where upon completion of the plan portion of the SOAP note, treatment plan data comprising the prescribed treatment plan is synced to, or stored in, a treatment panel of the EMR.

10. The electronic medical record system of claim 9 wherein said treatment panel comprises (i) a modalities panel comprising data regarding prescribed modalities and (ii), an exercise panel comprising data regarding prescribed tests.

11. The electronic medical record system of claim 10 including means for (1) indicating when modalities and/or exercises have been completed and (2) means for tagging said modalities and/or exercises completed in-office with a billing indicator; whereby said system is adapted to provide said billing indicator to a billing system which can generate a claim to be submitted to an insurance company and/or an invoice to be provided to the patient.

12. A method for generating a SOAP note in an electronic medical record system, the electronic medical record system comprising a patient portal, a practitioner portal, a server, and a non-volatile memory in which is stored an electronic medical record (EMR) data structure and a condition reference data structure, said condition reference data structure comprising data regarding diagnoses, care plans, treatment modalities, exercises, and tests; said patient portal and practitioner portal both being in communication with said server and being adapted to send information to, and receiving information from, said server;
said system including instructions stored in said non-volatile memory for generating a SOAP note, said SOAP note comprising a subjective portion, assessment portion, objective portion, and plan portion; said method including:
a) determining if the patient encounter is a first patient encounter or a subsequent patient encounter for the patient complaint;
b) generating a patient questionnaire and displaying said questionnaire on said patient portal; said step of generating the patient questionnaire comprising:
b1) for an initial visit for a patient complaint, providing the patient with an image of a body and enabling the patient to indicate which portion of the body is involved in the patient complaint, and generating further questions to the patient in response to answers received from the patient; and
b2) for a second or subsequent visit for the same complaint, generating questions for patient as a function of information from a prior SOAP note for the patient complaint, wherein said questions generated illicit information from the patient regarding the status of the patient complaint;
c) receiving responses from said patient portal to questions in said questionnaire entered by a patient;
d) populating the subjective portion of the SOAP note with the patient responses to the questionnaire;
e) upon request, displaying the subjective portion of the SOAP note on the practitioner portal;
f) receiving a diagnosis from the practitioner and entering the diagnosis in the assessment portion of the SOAP note;
g) auto-populating a care plan in the assessment portal based on the diagnosis and care plan data in the condition reference data structure;
h) auto-populating the objective portion of the SOAP note with proposed tests for the practitioner to run and displaying the proposed tests on the practitioner portal, the proposed tests being selected based on the diagnosis and care plan data in the condition reference data structure;
i) receiving from the practitioner portal test result information entered into the practitioner portal by the practitioner for tests run and storing test data in the objective portion of the SOAP note; said test data comprising tests run and test results for the tests run; said tests run including at least a portion of the tests proposed by the system;
j) auto-populating the plan portion of the SOAP note with a proposed plan of treatment for the patient and displaying the proposed plan of treatment on the practitioner portal; the proposed plan of treatment being based on the diagnosis, the test results, and care plan data in the condition reference data structure;
k) receiving from the practitioner portal information regarding a prescribed treatment plan and storing said prescribed treatment plan in the plan portion of the SOAP note; said prescribed treatment plan comprising at least in part aspects of the proposed treatment plan;
l) generating a completed SOAP note from the information stored int eh subjective portion, assessment portion, objective portion, and plan portion of the SOAP note and displaying the completed SOAP note on the practitioner portal;
m) enabling the practitioner to electronically sign the completed SOAP note;
n) upon receipt of an electronic signature for the SOAP note, storing the SOAP note as a SOAP note record for the particular patient encounter in an electronic medical record for the patient.

13. The method of claim 12 including steps of:
determining if a vital statistic (such as blood pressure, weight, BMI, pulse, etc.) is abnormal, generating a warning regarding said abnormal vital statistic, and displaying said warning on all screens of an electronic medical record for the patient; or
enabling the provider to enter an alert regarding a condition of the patient and displaying said warning on all screens of the electronic medical record for the patient.

14. The method of claim 12 wherein including a step of enabling the practitioner to request additional tests; wherein said test data includes said additional tests and results of said additional tests.

15. The method of claim 12 wherein said proposed plan of treatment comprises one or more of proposed imaging and/or tests, proposed treatments, proposed modalities, proposed supplements and/or supplies, and proposed exercises; wherein said method includes a step of enabling the practitioner to prescribe additional imaging and/or tests, treatments, modalities, supplements and/or supplies, and/or exercises not proposed by the system; wherein the prescribed plan of action comprises at least a portion of the proposed imaging and/or tests, proposed treatments, proposed modalities, proposed supplements and/or supplies, and proposed exercises and the additional prescribed imaging and/or tests, treatments, modalities, supplements and/or supplies, and exercises.

16. The method of claim 12 including a step of, upon completion of the objective portion of the SOAP note, syncing or storing test data comprising tests run and test results, to or in a test panel; said test panel containing a history of tests run on the patient.

17. The method of claim 12, where upon completion of the plan portion of the SOAP note, the method includes a step of syncing or storing treatment plan data comprising the prescribed treatment plan to or in a treatment panel.

18. The method of claim 17 wherein said treatment panel comprises (i) a modalities panel comprising data regarding prescribed modalities and (ii), an exercise panel comprising data regarding prescribed tests.

19. The electronic medical record system of claim 18 including a step of (1) indicating when modalities and/or exercises have been completed and (2) tagging said modalities and/or exercises completed in-office with a billing indicator; whereby said system is adapted to provide said billing indicator to a billing system which can generate a claim to be submitted to an insurance company and/or an invoice to be provided to the patient.

20. A computer-implemented electronic medical record system for automatically generating, storing, and securing a SOAP note for a patient encounter, the system comprising:
a. a server comprising at least one processor and a non-transitory computer-readable medium;
b. a patient portal and a practitioner portal, each being adapted to communicate with the server to exchange data with the server over a communication network;
c. the non-transitory computer-readable medium storing:
(i) an electronic medical record (EMR) data structure that, for each patient, includes at least one encounter record, each encounter record having at least a subjective portion, an assessment portion, an objective portion, and a plan; and
(ii) a condition reference data structure comprising machine-readable relational links that associate diagnoses with corresponding care-plan elements, clinical tests, therapeutic modalities, and exercise prescriptions; and
d. executable instructions which, when executed by the processor, cause the server to:
(1) prior to a first encounter for a particular patient complaint, transmit to the patient portal a dynamically generated electronic questionnaire that is iteratively built in real time in response to patient interaction with an anatomical graphical user interface, the questionnaire being restricted to questions pertinent to a body region selected by the patient;
(2) receive patient responses from the patient portal and automatically store the responses in the subjective portion of a newly created encounter record within the EMR data structure;
(3) render for display on the practitioner portal the stored subjective portion together with provisional assessment data items automatically retrieved from the condition reference data structure by matching the patient responses to linked diagnoses;
(4) receive final assessment data via the practitioner portal and store the final assessment data in the assessment portion of the encounter record, said final assessment data comprising the provisional assessment data if approved by the practitioner or practitioner-edited assessment data;
(5) automatically compile, based on the final assessment data and the relational links in the condition reference data structure, a set of proposed objective clinical tests ordered according to diagnostic utility, transmit the set to the practitioner portal, receive entered test results, and store the results in the objective portion of the encounter record;
(6) automatically generate, using the stored assessment data and objective test results, an individualized treatment plan comprising one or more of an imaging order, a therapeutic modality, and an exercise prescription, the treatment plan being derived by traversing the relational links of the condition reference data structure, and store the treatment plan in the plan portion of the encounter record;
(7) generate, within the EMR data structure and without user transcription, a structured electronic SOAP note that orders the stored subjective, assessment, objective, and plan portions sequentially as Subjective-Assessment-Objective-Plan;
(8) display the generated SOAP note on the practitioner portal for electronic signature; and
(9) for each subsequent encounter relating to the same patient complaint, automatically initiate a new encounter record that imports, as default values, data from at least the assessment and plan portions of a most recent prior encounter record, repeats operations (1)-(8) using updated questionnaire responses, and thereby minimizes redundant data entry across encounters.

21. A computer-implemented electronic medical record system for automatically generating, storing, and securing a SOAP note for a patient encounter, the system comprising:
a. a server comprising at least one processor and a non-transitory computer-readable medium;
b. a patient portal and a practitioner portal, each being adapted to communicate with the server to exchange data with the server over a communication network;
c. the non-transitory computer-readable medium storing:
(i) an electronic medical record (EMR) data structure that, for each patient, includes at least one encounter record, each encounter record having at least a subjective portion, an assessment portion, an objective portion, and a plan portion; and
(ii) a large language model (LLM) trained on medical data and capable of associating diagnoses with corresponding care-plan elements, clinical tests, therapeutic modalities, and exercise prescriptions; and
d. executable instructions which, when executed by the processor, cause the server to:
(1) prior to a first encounter for a particular patient complaint, transmit to the patient portal a dynamically generated electronic questionnaire that is iteratively built in real time in response to patient interaction with an anatomical graphical user interface, the questionnaire being restricted to questions pertinent to a body region selected by the patient;
(2) receive patient responses from the patient portal and automatically store the responses in the subjective portion of a newly created encounter record within the EMR data structure;
(3) render for display on the practitioner portal the stored subjective portion together with provisional assessment data items automatically generated by the large language model (LLM) by analyzing the patient responses and suggesting linked diagnoses;
(4) receive practitioner-edited assessment data via the practitioner portal and store the edited assessment data in the assessment portion of the encounter record;
(5) automatically compile, based on the edited assessment data and the output of the large language model (LLM), a set of proposed objective clinical tests ordered according to diagnostic utility, transmit the set to the practitioner portal, receive entered test results, and store the results in the objective portion of the encounter record;
(6) automatically generate, using the stored assessment data and objective test results, an individualized treatment plan comprising one or more of an imaging order, a therapeutic modality, and an exercise prescription, the treatment plan being derived by querying the large language model (LLM), and store the treatment plan in the plan portion of the encounter record;
(7) generate, within the EMR data structure and without user transcription, a structured electronic SOAP note that orders the stored subjective, assessment, objective, and plan portions sequentially as Subjective-Assessment-Objective-Plan;
(8) display the generated SOAP note on the practitioner portal for electronic signature, and, upon receipt of the signature, cryptographically lock the encounter record against further alteration; and
(9) for each subsequent encounter relating to the same patient complaint, automatically initiate a new encounter record that imports, as default values, data from at least the assessment and plan portions of a most recent prior encounter record, repeats operations (1)-(8) using updated questionnaire responses, and thereby minimizes redundant data entry across encounters.
